(19) United States
(12) Patent Application Publication
(10) Pub. No.: US 2021/0236824 A1
(43) Pub. Date: Aug. 5, 2021

(54) SYSTEMS AND METHODS FOR ELIMINATING ONSET RESPONSE IN NERVE CONDUCTION BLOCK

(71) Applicant: DUKE UNIVERSITY, Durham, NC (US)

(72) Inventor: Warren Grill, Durham, NC (US)

(21) Appl. No.: 17/167,579

(22) Filed: Feb. 4, 2021

Related U.S. Application Data
(60) Provisional application No. 62/970,303, filed on Feb. 5, 2020.

(51) Int. Cl. A61N 1/36; A61N 1/372

(52) U.S. Cl. CPC A61N 1/36192; A61N 1/37252; A61N 1/36153

(57) ABSTRACT

The present disclosure provides systems and methods relating to neuromodulation. In particular, the present disclosure provides systems and methods for eliminating the onset response when blocking nerve conduction. The various embodiments disclosed herein include methods for designing waveforms that block nerve conduction without inducing an onset response, and systems for delivering treatment based on these waveforms to subjects with pathological neural activity.

### RELATED APPLICATIONS

The This application claims priority to and the benefit of U.S. Provisional Patent Application No. 62/970,303 filed Feb. 12, 2020, which is incorporated herein by reference in its entirety for all purposes.

### GOVERNMENT FUNDING

This invention was made with government support under Federal Grant No. OT2 OD025340 awarded by the National Institutes of Health. The Federal Government has certain rights to the invention.

### FIELD

The present disclosure provides systems and methods relating to neuromodulation. In particular, the present disclosure provides systems and methods for eliminating the onset response when blocking nerve conduction. The various embodiments disclosed herein include methods for designing waveforms that block nerve conduction without inducing an onset response, and systems for delivering treatment based on these waveforms to subjects with pathological neural activity.

### BACKGROUND

Many neurological disorders, including pain and spasticity, are characterized by undesirable increases in sensory, motor, or autonomic nerve activity. Local application of kilohertz frequency alternating currents (KHFAC) can effectively and completely block the conduction of undesired hyperactivity through peripheral nerves and could be a therapeutic approach for alleviating disease symptoms. However, KHFAC nerve block produces an undesirable initial burst of action potentials (APs) prior to achieving block, known as onset response. This onset firing may result in muscle contraction and pain and is a significant impediment to potential clinical applications of KHFAC nerve block. There are some existing approaches to reduce or eliminate the onset response, such as electrode geometry design and amplitude and frequency modulation of the applied signal. However, these approaches are often unable to completely eliminate the onset firing and can be challenging for clinical implementation. Hence, there is an ongoing need for improved methods of KHFAC treatment.

### SUMMARY

Embodiments of the present disclosure include a method of designing a waveform shape for blocking neural conduction without inducing an onset response. In accordance with these embodiments, the method includes identifying an optimized transmembrane voltage trajectory sufficient to induce closed-state inactivation (CSI) in a plurality of voltage-gated sodium channels, identifying at least one DC blocking waveform from a plurality of candidate waveforms using a global optimization algorithm, and designing a waveform that blocks neural conduction without inducing an onset response based on the at least one DC blocking waveform. In some embodiments, the at least one DC blocking waveform drives a voltage profile corresponding to the optimized transmembrane voltage trajectory.

In some embodiments, the waveform that blocks neural conduction without inducing an onset response is an AC blocking waveform. In some embodiments, the AC blocking waveform comprises an amplitude envelope defined by the least one DC blocking waveform.

In some embodiments, the optimized transmembrane voltage trajectory is identified using a computation model comprising characteristics of a nerve fiber. In some embodiments, the characteristics of the nerve fiber are selected from the group consisting of number of nodes of Ranvier, nerve fiber diameter, nerve fiber length, nerve fiber location, types of sodium channels, number of sodium channels, types of potassium channels, number of potassium channels, and degree of myelination.

In some embodiments, the optimized transmembrane voltage trajectory is identified using a global optimization algorithm. In some embodiments, the global optimization algorithm is selected from the group consisting of a genetic algorithm, a particle swarm algorithm, a simulated annealing algorithm, an ant colony algorithm, an estimation of distribution algorithm, and any combinations and derivations thereof.

In some embodiments, the global optimization algorithm used to identify the at least one DC blocking waveform is selected from the group consisting of a genetic algorithm, a particle swarm algorithm, a simulated annealing algorithm, an ant colony algorithm, an estimation of distribution algorithm, and any combinations and derivations thereof. In some embodiments, the optimized transmembrane voltage trajectory increases nonlinearly from a resting potential to a suprathreshold level.

In some embodiments, the AC blocking waveform is applied at increasing amplitude over time. In some embodiments, the AC blocking waveform is charge-balanced. In some embodiments, the AC blocking waveform is biphasic, monophasic, or multiphasic. In some embodiments, the AC blocking waveform is applied at a frequency of about 5 kHz to about 90 kHz. In some embodiments, the AC blocking waveform is symmetric and/or rectangular.

Embodiments of the present disclosure also include an AC conduction block waveform that eliminates onset response. In accordance with these embodiments, the waveform is applied at increasing amplitude over time, and the waveform blocks neural conduction and induces closed-state inactivation (CSI) of voltage-gated sodium channels prior to activation.

Embodiments of the present disclosure also include a system for blocking neural conduction without inducing an onset response. In accordance with these embodiments, the system includes an electrode sized and configured for implantation in proximity to neural tissue, and a pulse generator coupled to the electrode. In some embodiments, the pulse generator includes a power source comprising a battery and a microprocessor coupled to the battery. In some embodiments, the pulse generator is capable of applying to the electrode an AC waveform capable of blocking neural conduction and eliminating onset response by inducing closed-state inactivation (CSI) of voltage-gated sodium channels prior to activation.

In some embodiments, the AC blocking waveform is applied at increasing amplitude over time. In some embodiments, the AC blocking waveform is charge-balanced. In some embodiments, the AC blocking waveform is biphasic, monophasic, or multiphasic. In some embodiments, the AC blocking waveform is applied at a frequency of about 5 kHz to about 90 kHz. In some embodiments, the AC blocking waveform is symmetric and/or rectangular.

Embodiments of the present disclosure also include a method for blocking neural conduction in a subject using any of the systems described herein. In accordance with these embodiments, the method includes programming the pulse generator to output the blocking waveform at increasing amplitude over time, and delivering the blocking waveform to the subject without inducing an onset response.

### BRIEF DESCRIPTION OF THE DRAWINGS

FIGS. 1A-1B: Hybrid model of a peripheral myelinated axon incorporating Markov-type VGSCs. (A) Schematic of the model, which included 21 nodes of Ranvier and 20 internodal segments. In the original MRG model, each node included fast Na⁺ (I_{Naf}), persistent Na⁺ (I_{Nap}), slow K⁺ (I_{KS}), and linear leakage (I_{L}) currents, and membrane capacitance (Cₙ), and the ionic currents were described using HH-type kinetics. I_{Naf} and I_{Nap} were substituted in node 6 to node 14 with Markov-type Naᵥ 1.1 (I_{Nav11}) and Naᵥ 1.6 (I_{Nav16}) channels, respectively. The internodal segments used a double-cable concentric structure to represent the myelin attachment segment (MYSA), paranode main segment (FLUT), and internode segment (STIN). The internodal segments were modeled with linear conductances (Gₘ, Gᵢ) and membrane capacitances (Cₘ, Cᵢ). Adjacent segments were connected by axoplasmic (Gₐ) and periaxonal conductances (Gₚ). Resting membrane potential Vᵣₑₛₜ was −80 mV, and the fiber diameter was 10 μm. The axon-specific electrical and geometrical parameters followed McIntyre et al. and are provided in Tables 1 and 2. (B) Schematic of the states and transitions of Markov-type VGSCs. C1 and C2 were closed states, O1 and O2 were open states, I1 and I2 were inactivation states, and A_{w1w2} was the transition rate from state w1 to state w2. Pink lines represented a CSI path, and blue lines represented an OSI path. The parameters of the transition rate for Naᵥ 1.1 and Naᵥ 1.6 channels are provided in Table 3.

FIGS. 2A-2F: Trajectory of transmembrane voltage to cause Na⁺ CSI. (A) Top panel: Voltage step profile Vₛₜₑₚ. The transmembrane voltage was increased from the resting potential of −80 mV to −10 mV at 25 ms. Center panel: Naᵥ 1.1 current during Vₛₜₑₚ. Bottom: Naᵥ 1.6 current during Vₛₜₑₚ. (B) Top panel: fractions of Naᵥ 1.1 channels in states C1 (C11), I1 (I11), I2 (I12), and O1 (O11) during Vₛₜₑₚ. Bottom panel: fractions of Naᵥ 1.6 channels in states C1 (C61), I1 (161), I2 (I62), and O1 (O61) during Vₛₜₑₚ. (C) Voltage response recorded in each node when applying Vₛₜₑₚ at node 10. (D) Top panel: PSO-generated optimized voltage profile V_{PSO}. Center panel: Naᵥ 1.1 current during V_{PSO}. Bottom panel: Naᵥ 1.6 current during V_{PSO}. (E) Top panel: C11, I11, I12, and O11 of Naᵥ 1.1 channels during V_{PSO}. Bottom panel: C61, I61, I62, and O61 of Naᵥ 1.6 channels during V_{PSO}. (F) Voltage response recorded in each node when applying V_{PSO} as a series of voltage clamps at node 10.

FIGS. 3A-3G: PSO-generated DC waveform drives V_{PSO} and generates onset-free conduction block. (A) Simulation setup. A monopolar block electrode was placed 1 mm over node 10, and suprathreshold intracellular test pulses were delivered at node 0 to generate propagating APs. (B) Top panel: Voltage response recorded at node 10 (red solid line) and the PSO-generated voltage profile V_{PSO} (black dotted line). Bottom panel: PSO-generated DC waveform I_{PSO} for driving V_{PSO} in node 10. (C) Voltage response recorded in each node after applying I_{PSO}. No test pulse was delivered at node 0. (D) Propagation or block of a single test AP along the axon initiated at a time delay Δτ after the onset of I_{PSO}. Top panel: Δτ=35 ms, bottom panel: Δτ=37 ms. (E) Block of train of 50 Hz test APs by I_{PSO}. (F) Block of train of 100 Hz test APs by I_{PSO}. (G) Block of train of 200 Hz test APs by I_{PSO}.

FIGS. 4A-4C: PSO-based charge-balance biphasic KHFAC waveform for onset-free conduction block. (A) Transmembrane voltage (top) and Naᵥ 1.1 current I_{Nav11} (center) recorded in node 10 in response to KHFAC current I_{BI} (bottom). The frequency of I_{BI} was 10 kHz, and each pulse was rectangular, symmetric, and charge-balanced. The envelope of I_{BI} was determined by multiplying I_{PSO} (red line) by a scale factor of 1.5. (B) Fractions of Naᵥ 1.1 channels in the states C1 (C11), I2 (I12), and O1 (O11). (C) Voltage response recorded in each node after applying I_{BI} with a monopolar electrode placed 1 mm above the central node. No test pulse was injected at node 0. The red lines in (A) and (B) were the responses associated with DC waveform I_{PSO}.

FIGS. 5A-5E: Onset-free conduction block by PSO-based KHFAC waveform. (A) Propagation or block of a single test AP along the axon initiated at a time delay Δτ after the onset of I_{BI}. Top panel: Δτ=47 ms, bottom panel: Δτ=49 ms. A scale factor of 1.5 was used to design the envelope of I_{BI}. (B) Minimum time delay Δτₘᵢₙ for producing conduction block as a function of scale factor. (C) Block of train of 50 Hz test APs by I_{BI}. (D) Block of train of 100 Hz test APs by I_{BI}. (E) Block of train of 200 Hz test APs by I_{BI}. PSO-based KHFAC waveform I_{BI} was applied using a monopolar block electrode placed over the central node at an electrode-fiber distance of 1 mm.

FIGS. 6A-6B: Flowchart of the PSO algorithm for (A) designing a transmembrane voltage trajectory for causing Na⁺ CSI and (B) generating DC waveform for driving that trajectory.

FIGS. 7A-7B: Setting maximum conductance of Naᵥ 1.1 channels. Propagation of a test AP initiated in node 0 along the 21-node and 10 μm model at (A) g̅_{Nav11}=4.7 S/cm² and (B) 11.9 S/cm². g̅_{Nav11}=4.7 S/cm² was the minimum conductance for faithfully propagating a single AP along the axon, and g̅_{Nav11}=11.9 S/cm² was the minimum conductance for faithfully propagating spike trains at rates of up to 400 Hz along the axon. I_{Naf} was recorded in node 3, and I_{Nav11} was recorded in node 10. Left panels: a single test pulse (width: 0.1 ms and amplitude: 2.5Iₜₕ) was delivered at the node 0. Right panels: 200 Hz test pulses were delivered at the node 0. Maximum conductance of HH-type I_{Naf} was g̅_{Naf}=3.0 S/cm², maximum conductance of HH-type I_{Nap} was g̅_{Nap}=0.01 S/cm², and maximum conductance of Markov-type I_{Nav16} was g̅_{Nav16}=0.01 S/cm².

FIGS. 8A-8C: Conduction block at electrode-fiber distance of 0.3 mm by PSO-based KHFAC waveform. (A) Simulation setup. A monopolar block electrode was placed 0.3 mm over the central node of 21-node 10 μm diameter model nerve fiber. An intracellular test pulse (width: 0.1 ms and amplitude: 2.5Iₜₕ) was delivered at node 0 to generate a propagating AP at t=120 ms. (B) Transmembrane voltages (top) recorded in node 0 and node 20 in response to KHFAC waveform I_{BI} (bottom). A scale factor of 1.5 was used to design the envelope of and the green dotted line was the block threshold at electrode-fiber distance of 0.3 mm. (C) Voltage responses recorded in node 10 to node 14.

FIGS. 9A-9C: Effects of fiber diameter on conduction block by PSO-based KHFAC waveform. (A) Simulation setup. A monopolar block electrode was placed 1 mm over node 125 and delivered KHFAC waveform I_{BI} to a 251-node model. An intracellular test pulse (width: 0.1 ms and amplitude: 2.5Iₜₕ) was delivered at node 0 to generate a propagating AP at t=120 ms. Markov-type Naᵥ 1.1 and Naᵥ 1.6 channels were implemented in node 121 to node 129. (B) Block of a test AP along the axon by I_{BI} with a fiber diameter of 2.0 μm. (C) Block of a test AP along the axon by I_{BI} with a fiber diameter of 8.7 μm. In (B) and (C), the green dotted lines were the block threshold at each fiber diameter.

FIGS. 10A-10B: Conduction block in HH-type model by PSO-based KHFAC waveform. (A) Block of a test AP along the 21-node 10 μm diameter model nerve fiber by PSO-based waveform I_{BI}. No Markov-type VGSCs were implemented in the central nodes. The block electrode was placed 1 mm above node 10, and a single test pulse (width: 0.1 ms and amplitude: 2.5Iₜₕ) was delivered at node 0 to generate a propagating AP at t=70 ms. (B) Top panel: transmembrane voltage recorded in node 10 in response to KHFAC waveform I_{BI}. Center panel: activation gating variable m and inactivation gating variable h of fast Na⁺ current. Bottom panel: PSO-based KHFAC waveform I_{BI} with a plateau amplitude scaled to the block threshold.

FIGS. 11A-11C: Effects of number of Markov-type nodes on conduction block by PSO-based KHFAC waveform. (A) Simulation setup. Naᵥ 1.1 and Naᵥ 1.6 channels were implemented in node (10−n) to node (10+n), where 0≤n≤10. A monopolar block electrode was placed 1 mm over node 10, and an intracellular test pulse (width: 0.1 ms and amplitude: 2.5Iₜₕ) was delivered at node 0 to generate a propagating AP at t=120 ms. (B) Block threshold as a function of the number of Markov-type nodes. At 3≤n≤10, PSO-based KHFAC waveform I_{BI} produced onset-free conduction block. At 0≤n≤2, no conduction block occurred and onset firing was activated by I_{BI}. (C) Propagation of a test AP along the axon by I_{BI} (n=1). A scale factor of 1.0 was used to design the envelope of I_{BI}, and the number of Markov-type nodes was three.

FIGS. 12A-12B: PSO-based KHFAC waveform designed using bipolar and tripolar cuff electrodes. (A) The extracellular potential was interpolated at each segment of 21-node hybrid model from a finite element model (FEM) of the rat tibial nerve, which had a bipolar nerve cuff and a model nerve fiber that was 0.398 mm away from the electrode. Since the bipolar cuff resulted in the largest depolarization in node 11, a PSO algorithm was applied to generate a DC waveform I_{PSO} to drive the optimized voltage trajectory V_{PSO} in node 11. The envelope of KHFAC waveform I_{BI} was determined by multiplying I_{PSO} by a scale factor of 0.73, and the plateau amplitude of I_{BI} was the block threshold. The resulting KHFAC waveform blocked nerve conduction with activation of an onset response. (B) An FEM of a tripolar cuff was used on the rat tibial nerve to interpolate the extracellular potentials of 21-node hybrid model, and the electrode-fiber distance was 0.398 mm. The DC waveform I_{PSO} was designed to drive V_{PSO} in node 10. A scale factor of 0.93 was applied to scale the plateau amplitude of I_{BI} to the block threshold. The resulting KHFAC waveform produced onset-free conduction block. A single test pulse (width: 0.1 ms and amplitude: 2.5Iₜₕ) was injected in node 0 at t=70 ms. Fiber diameter was 10 μm.

FIGS. 13A-13C: Progression of PSO for generating transmembrane voltage trajectory to cause Na⁺ CSI. (A) Changes of transmembrane voltage trajectory across iterations. The sequence of plots show the voltage profile with the minimum cost ƒₜ at each indicated iteration. (B) Minimum cost ƒₜ of 50 particles at each iteration. Red dotted line is 1.01 times the minimum cost at the final generation. (C) Voltage response recorded in each node when applying the voltage profile at each indicated iteration as a series of voltage clamps at node 10. Fiber diameter was 10 μm.

FIG. 14: Minimum cost function ƒ₂ of 50 particles at each iteration. Red dotted line was 1.01 times the minimum cost at the final generation.

### DETAILED DESCRIPTION

The present disclosure provides systems and methods relating to neuromodulation. In particular, the present disclosure provides systems and methods for eliminating the onset response when blocking nerve conduction. The various embodiments disclosed herein include methods for designing waveforms that block nerve conduction without inducing an onset response, and systems for delivering treatment based on these waveforms to subjects with pathological neural activity.

In accordance with these embodiments, the present disclosure provides optimized waveforms for administering nerve blocking in a subject, and method for designing optimized waveforms. In some embodiments, the present disclosure provides a novel engineering optimization approach for designing a blocking waveform to completely eliminate onset firing in peripheral axons by moving voltage-gated Na⁺ channels (VGSCs) to closed-state inactivation (CSI). The resulting KHFAC waveforms of the present disclosure can generate electric nerve block without inducing an onset response. The methods and systems provided herein for optimizing blocking waveforms represents a novel engineering design methodology with myriad potential applications and has relevance for the conduction block of peripheral nerve hyperactivity.

As described further herein, a hybrid axon model was created to develop and test candidate waveforms. In one embodiment, a Markov-type kinetic model was used to simulate Na⁺ channel CSI; this model was previously validated with electrophysiological data from all VGSC isoforms. In another embodiment, a McIntyre-Richardson-Grill (MRG) model of a mammalian peripheral myelinated axon was modified to incorporate Markov-type Naᵥ 1.1 and Naᵥ 1.6 channels. In accordance with these embodiments, the methods and systems of the present disclosure include the use of such models to design and test optimized waveforms using various input parameters, such as electrode type, axon diameter, electrode position, and the like. In some embodiments, an optimized waveform includes waveforms that maximize the number of VGSCs in a CSI state so as to eliminate the onset response resulting from KHFAC block.

In some embodiments, methods of the present disclosure include the use of a optimization algorithm to design an optimized transmembrane voltage trajectory to drive VGSCs to CSI without first causing activation of the VGSCs. In some embodiments, the same or similar algorithm can then be used to generate an extracellular DC stimulation waveform to drive the voltage profile to follow the optimized trajectory. In some embodiments, an AC waveform is generated based on the generated DC waveform and is administered to a subject using a neuromodulation system, for example, to treat pathological neural activity. In some embodiments, the optimization algorithm is a particle swarm optimization (PSO), and the method includes a multi-objective optimization to find a voltage trajectory that maximizes the fractions of Naᵥ 1.1 and Naᵥ 1.6 channels in inactivated states while minimizing the fractions of the two VGSCs in open states.

In some embodiments, after a voltage trajectory is identified, the optimization algorithm is used to generate a plurality of candidate waveforms for driving the optimized voltage trajectory in an axon. In some embodiments, the optimization algorithm (e.g., PSO) is again applied, with the particles defined as the electrode currents at each time step, to design an extracellular stimulation waveform to drive PSO-generated voltage profile. In this case, the goal of the PSO is to minimize the difference between the membrane voltage recorded in a particular node and the optimized voltage profile. In some embodiments, this can be applied to a single axon, or two or more axons (e.g., a population of axons). In some embodiments, the waveform can be designed for driving the voltage profile in a group of axons, where the axons may have different properties, including but not limited to, diameter and distance from the electrode.

Results provided herein demonstrate that a PSO-based KHFAC waveform produces robust conduction block without activating an onset response. Thus, it is anticipated that the disclosed waveform can be useful in applying a therapeutic nerve block to a subject. It is additionally noted that the example PSO-based KHFAC waveform completely prevented the onset firing in the axon model for a prescribed set of parameters. For example, as described further herein, the electrode was assumed to be at a specific distance from a nerve fiber, and that the nerve fiber had a specific diameter. However, it is within the scope of the disclosure to design an optimized waveform based on different values of these and other input parameters.

Embodiments of the present disclosure also include a method of using a computationally optimized stimulation waveform to block neural activity in a subject without inducing an onset response. In some embodiments, the waveform can be delivered to the subject as an electrical stimulus using a neuromodulation device.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“About” is used to provide flexibility to a numerical range endpoint by providing that a given value may be “slightly above” or “slightly below” the endpoint without affecting the desired result.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Pain” generally refers to the basic bodily sensation induced by a noxious stimulus, received by naked nerve endings, characterized by physical discomfort (e.g., pricking, throbbing, aching, etc.) and typically leading to an evasive action by the individual. As used herein, the term pain also includes chronic and acute neuropathic pain. The term “chronic neuropathic pain” refers to a complex, chronic pain state that is usually accompanied by tissue injury wherein the nerve fibers themselves may be damaged, dysfunctional or injured. These damaged nerve fibers send incorrect signals to other pain centers. The impact of nerve fiber injury includes a change in nerve function both at the site of injury and areas around the injury. The term “acute neuropathic pain” refers to self-limiting pain that serves a protective biological function by acting as a warning of on-going tissue damage. Acute neuropathic pain is typically a symptom of a disease process experienced in or around the injured or diseased tissue.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

The term “effective amount” or “therapeutically effective amount” refers to an amount sufficient to effect beneficial or desirable biological and/or clinical results.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, neurobiology, microbiology, genetics, electrical stimulation, neural stimulation, neural modulation, and neural prosthesis described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. WAVEFORM OPTIMIZATION

Multiple neurological disorders are characterized by undesirable increases in sensory, motor, or autonomic nerve activity, for example pain and spasticity. Blocking the conduction of activity in nerves has the potential to alleviate disease symptoms. Kilohertz frequency alternating current (KHFAC) stimulation is an effective method for producing a rapid, controlled, and locally-acting conduction block. KHFAC nerve block is quickly reversible, and thus enables both temporal and spatial control of the target tissue with minimal side effects. However, an important drawback of KHFAC block is the transient burst of action potentials (APs) activated at the onset of blocking currents. This onset firing is likely to lead to intense muscle contraction and or painful sensations, and/or other unwanted physiological responses, and is a significant impediment to potential clinical applications of KHFAC nerve block. Several approaches are proposed to reduce or eliminate the onset response, including transitioning KHFAC waveform from a high amplitude and high frequency to a low amplitude and low frequency, slowly increasing the amplitude of the KHFAC waveform, introducing direct current (DC) nerve block, and modifying electrode geometries. However, these approaches are either unable to eliminate completely the onset firing or are challenging for clinical implementation. As described further herein, engineering optimization was used to design a stimulus waveform to achieve conduction block while preventing the onset responses.

Nerve excitation is regulated by voltage-gated Na⁺ channels (VGSCs). During an AP, the VGSCs first activate from non-conducting closed states to open states when membrane depolarization exceeds a threshold. The open channels allow influx of Na⁺ ions, which further depolarizes the cell and drives the upstroke of the AP. Membrane depolarization also causes inactivation of VGSCs, which prevents Na⁺ influx and facilitates repolarization to the resting potential. Na⁺ inactivation is coupled to activation, and typically occurs from the open state at strongly depolarized membrane potentials, also called open-state inactivation (OSI). However, recordings in cardiac cells, skeletal muscle, neuroblastoma cells, squid giant axon, medullary raphe neurons, and dorsal root ganglion neurons confirm that VGSCs can also inactivate from pre-open closed states without first opening, called closed-state inactivation (CSI). This path occurs at hyperpolarized and modestly depolarized potentials without generation of an AP.

Thus, as described further herein, experiments were conducted to investigate whether moving VGSCs into CSI without first causing activation could eliminate the initiation of APs and thus prevent the onset response. Embodiments of the present disclosure used Markov-type kinetic models to simulate Na⁺ channel CSI, and the models were previously validated with electrophysiological data from all VGSC isoforms. The McIntyre-Richardson-Grill (MRG) model of a mammalian peripheral myelinated axon was modified to incorporate Markov-type Naᵥ 1.1 and Naᵥ 1.6 channels. Additionally, particle swarm optimization (PSO) was used to design the profile of membrane voltage required to move the two types of VGSCs into CSI, and, subsequently, to determine the stimulation waveform shape required to drive that voltage profile. PSO is an effective search method for dealing with non-linear optimization problems by moving the particles to a “preferred” position in their constrained domains. It is effective for determining sequences of real numbers in non-linear systems, and was well suited to designing the voltage and current waveforms of the present disclosure; however, as would be recognized by one of ordinary skill in the art based on the present disclosure, other optimization algorithms can also be used. The outcome of the engineering optimization was a KHFAC current waveform that produced nerve conduction block without generating an onset response. The results provided herein revealed that moving VGSCs into the CSI without first opening completely suppressed the onset firing and demonstrated that using this novel approach for designing blocking waveforms may have relevance for the conduction block of peripheral nerve hyperactivity.

In accordance with these embodiments, a novel design process was used to develop a KHFAC stimulus waveform to enable nerve conduction block without the onset response that occurs with conventional KHFAC waveforms. In some embodiments, a charge-balanced 10 kHz biphasic waveform based on the PSO-generated DC waveform was designed, and this PSO-based KHFAC waveform produced robust conduction block without an onset response.

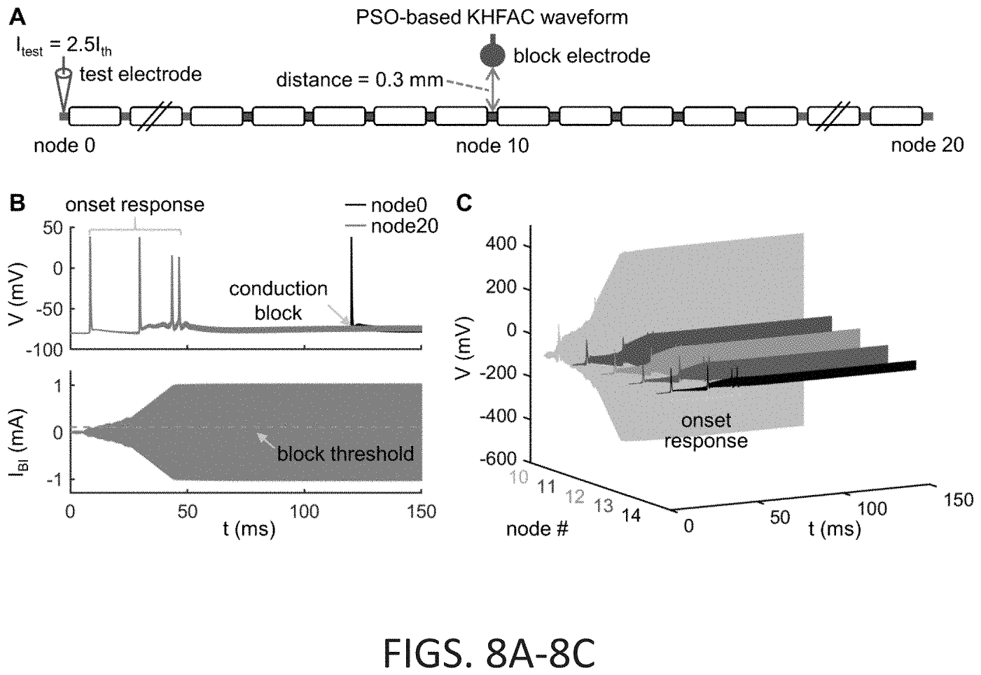

In in vivo experiments, a KHFAC waveform is commonly delivered using an extracellular electrode and can affect a number of axons with different electrode-fiber distances. The PSO-based biphasic waveform I_{BI} was calculated in a 21-node model at distance of 1 mm, and this waveform did not produce onset firing. The block threshold increases with increasing electrode-fiber distance, and it was examined whether the PSO-based KHFAC waveform optimized at a specific location produced onset-free conduction block in the axon at other locations. Applying I_{BI} with same plateau amplitude generated an onset response in the model at an electrode-fiber distance of 0.3 mm (FIG. 8). These closer nerve fibers had a block threshold lower than the plateau amplitude of I_{BI}, and as the KHFAC amplitude approached the plateau, the node underneath the blocking electrode was strongly depolarized and APs were initiated in adjacent nodes. While the PSO-based biphasic waveform eliminated the onset response in nerve fibers at certain locations, it may still activate onset firing at the other locations.

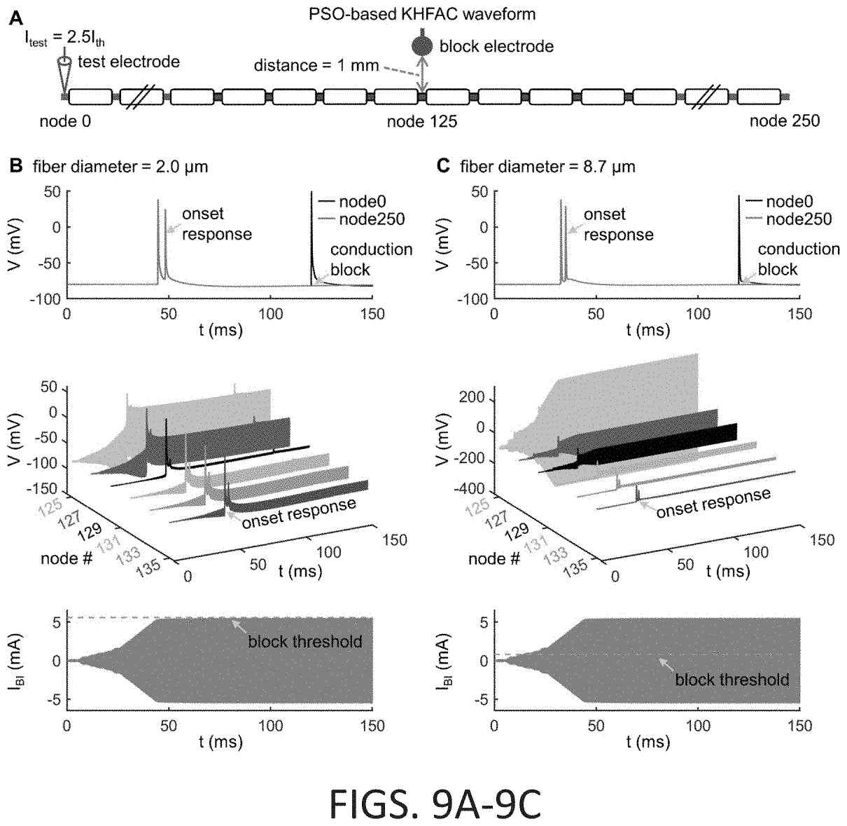

In in vivo experiments, a KHFAC waveform delivered using an extracellular electrode can also affect axons with different fiber diameters. The block threshold decreases with increasing fiber diameter, and it was examined whether the PSO-based KHFAC waveform produced onset-free conduction block in smaller diameter (2 μm and 8.7 μm) model axons. The internodal lengths of 2 μm or 8.7 μm axons are much shorter than those of the 10 μm fiber (Table 1), which significantly reduced the total length of the 21-node model axon. Since the electrode-fiber distance was a fixed value (1 mm), the number of nodes was increased to 251 to examine conduction block using the PSO-based KHFAC waveform and to avoid potential end effects, as the ends of the 21-node model axons with shorter intermodal lengths were much closer to the electrode. When scaling the plateau amplitude of I_{BI} to the block threshold of a 251-node model at a diameter of 2 μm, two APs were generated as the KHFAC amplitude approached the block threshold (FIG. 9). Applying I_{BI} with same plateau amplitude to a 251-node model at a diameter of 8.7 μm also produced an onset response. While the PSO-based KHFAC waveform eliminated onset response in the fibers for which it was designed, it may generate onset responses in other fiber diameters.

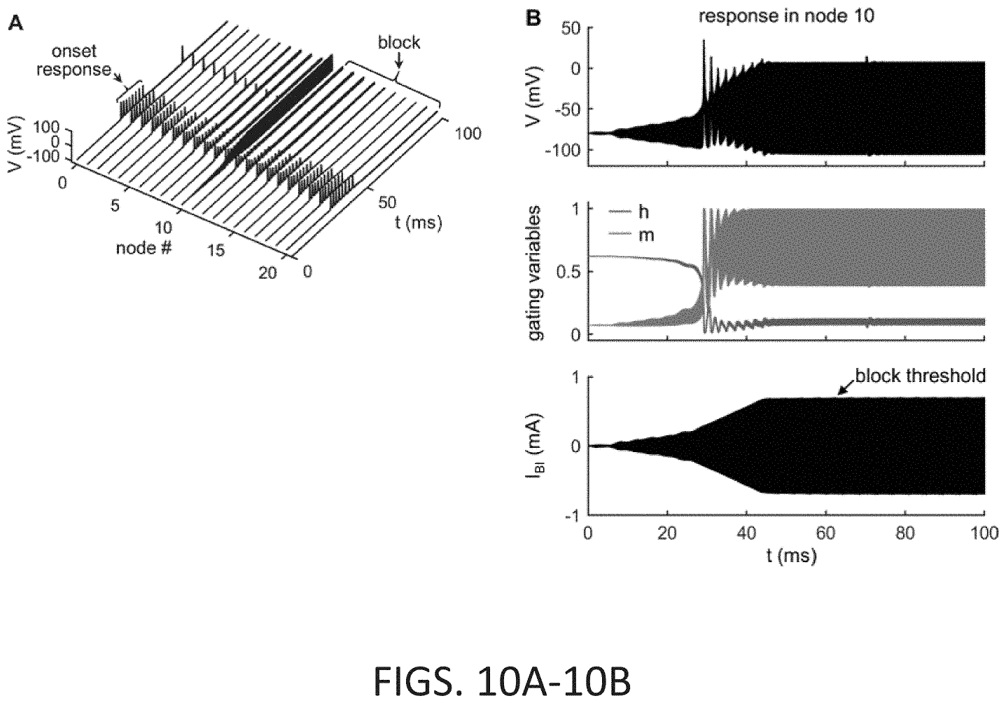

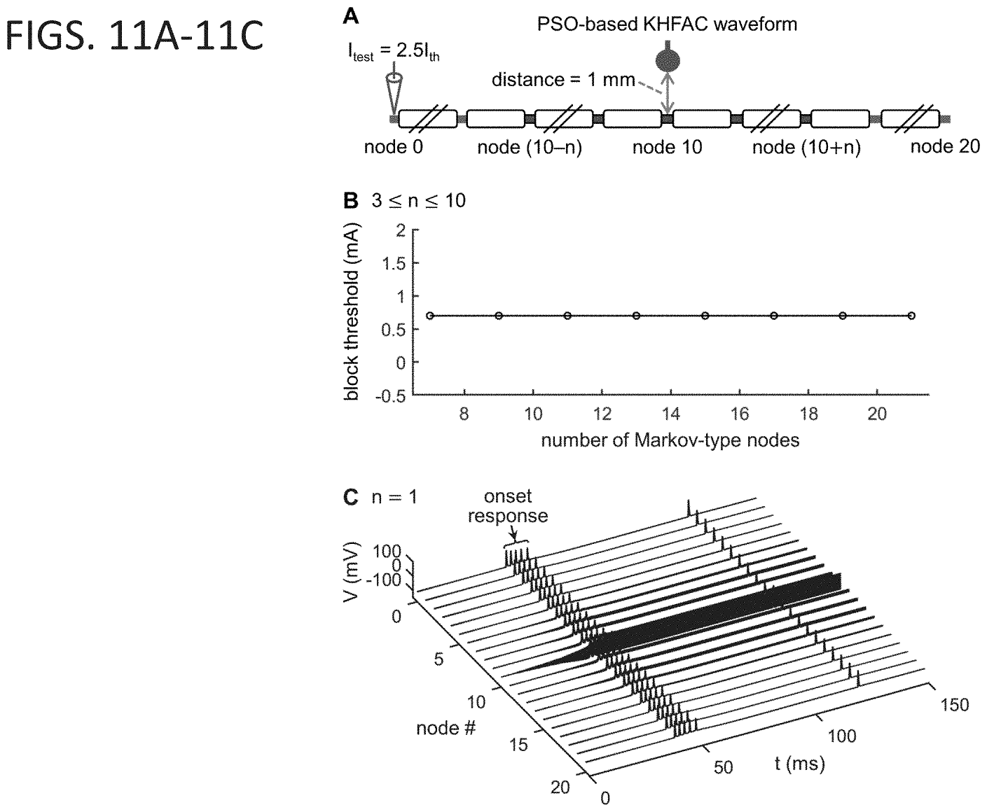

The original MRG models used the HH-type formalism to describe fast Na⁺ and persistent Na⁺ channels in each node, and the waveform was designed using the hybrid model incorporating Markov-type channels. Applying the PSO-based biphasic waveform to a 21-node 10 μm HH-type MRG axon (e.g., without the Markov-type VGSCs in the central nodes) generated eight APs as KHFAC amplitude approached the block threshold (FIG. 10). The initial ramp of biphasic stimulus drove the central node under the block electrode to suprathreshold depolarization, which enabled the initiation of onset firing. The VGSC gating variables m and h operate independently of each other, and no transition occurred between them as KHFAC amplitude increased. In contrast, the Markov-type Naᵥ 1.1 channels moved from the closed state to inactivation without fully opening in response to the PSO-based biphasic waveform, and thus no onset response was generated. To reduce overall computational demands, Naᵥ 1.1 and Naᵥ 1.6 channels were implemented only in the central nine nodes of a 21-node MRG model, but the specific number of Markov-type nodes did not alter the block threshold or the lack of an onset response in the hybrid model (FIG. 11). Additionally, as would be recognized by one of ordinary skill in the art based on the present disclosure, other computational models can also be used in conjunction with the methods and systems designed herein.

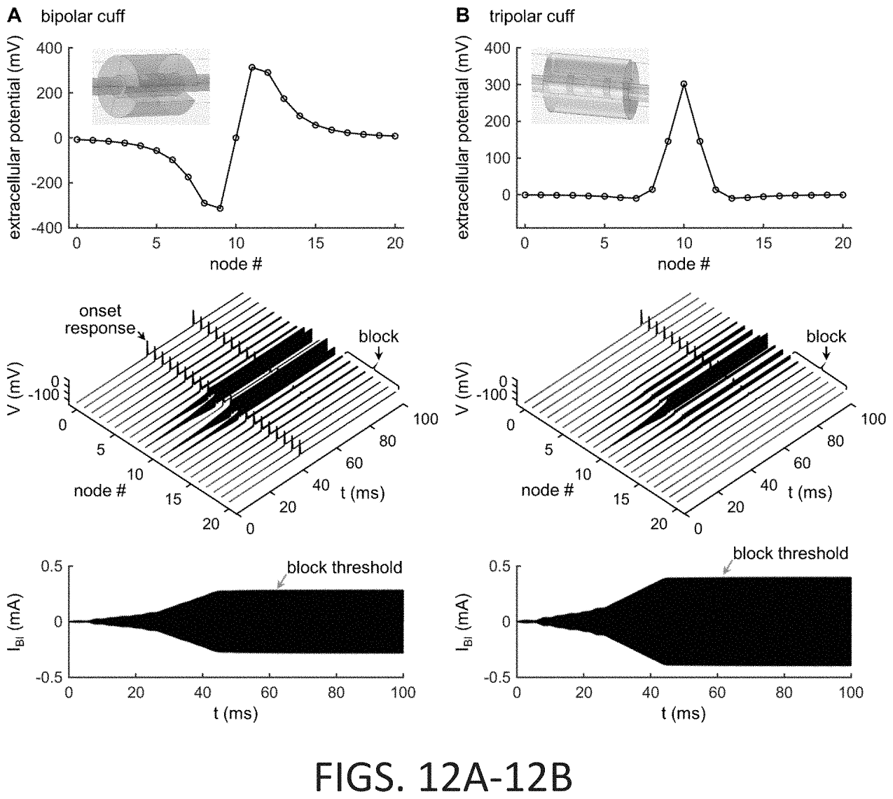

An earlier study of somatic voltage clamp reported that applying a voltage pre-pulse inactivated axonal channels after evoking an axonal spike, and the voltage steps following the pulse no longer triggered axonal spikes. The present disclosure showed that directly applying voltage clamp of the PSO-generated voltage profile V_{PSO} at the central node generated CSI of VGSCs, and no APs were escaped from the node. Further, a monopolar point source electrode was used to design PSO-based DC and KHFAC waveforms in the hybrid MRG model, while bipolar and tripolar cuff electrodes are commonly used in vivo. Linearly ramping KHFAC amplitude from zero to block threshold in rat sciatic nerve did not eliminate the onset response with a tripolar electrode, while linearly ramping KHFAC waveforms from non-zero amplitudes reduced the onset response with a bipolar electrode. Using representations of bipolar and tripolar cuff electrodes, the PSO-generated DC and biphasic waveforms were re-calculated, and the resulting KHFAC waveform was similar for each of three electrodes. Using a bipolar cuff, there was onset firing activated in the 21-node hybrid model as the KHFAC amplitude approached the block threshold (FIG. 12), but using the tripolar cuff, no onset response was generated by the initial ramp of the KHFAC waveform, as observed with the original monopolar electrode.

Other waveforms were proposed to eliminate the onset response. For example, Miles et al. showed that the KHFAC ramp that started from zero amplitude did not eliminate the onset response in either the HH-type MRG model or in experiments on rat sciatic nerve. Additionally, in vivo recordings demonstrated that the onset response was minimized with high amplitudes and high frequencies, and Gerges et al. showed that a transition from a high amplitude and high frequency to a low amplitude and low frequency during KHFAC nerve block minimized or eliminated the onset response with longer transition times. A DC block was also introduced to eliminate the onset response. Ackermann et al. reported that a brief DC block in combination with KHFAC prevented the onset firing, but such DC was found to produce nerve damage and prolonged conduction failure. Subsequently, Franke et al. suggested that combining KHFAC and charge-balanced DC block significantly reduced or completely prevented the onset response without a change in nerve conduction by using a block electrode of high capacitance materials.

Thus, embodiments of the present disclosure provide an engineering optimization method to design a charge-balanced biphasic current waveform that eliminates the onset response during KHFAC nerve block. Surprisingly, embodiments of the PSO-based KHFAC waveforms described herein completely eliminated or prevented the onset firing in axons at specific locations and with specific diameters, which demonstrates somewhat unexpectedly that it is possible to achieve KHFAC nerve block without an onset response by moving VGSCs to CSI.

In accordance with the above, embodiments of the present disclosure include a method of designing a waveform shape for blocking neural conduction without inducing an onset response. In some embodiments, the method includes identifying an optimized transmembrane voltage trajectory sufficient to induce closed-state inactivation (CSI) in a plurality of voltage-gated sodium channels, identifying at least one DC blocking waveform from a plurality of candidate waveforms using a global optimization algorithm, and designing a waveform that blocks neural conduction without inducing an onset response based on the at least one DC blocking waveform. In some embodiments, the at least one DC blocking waveform drives a voltage profile corresponding to the optimized transmembrane voltage trajectory.

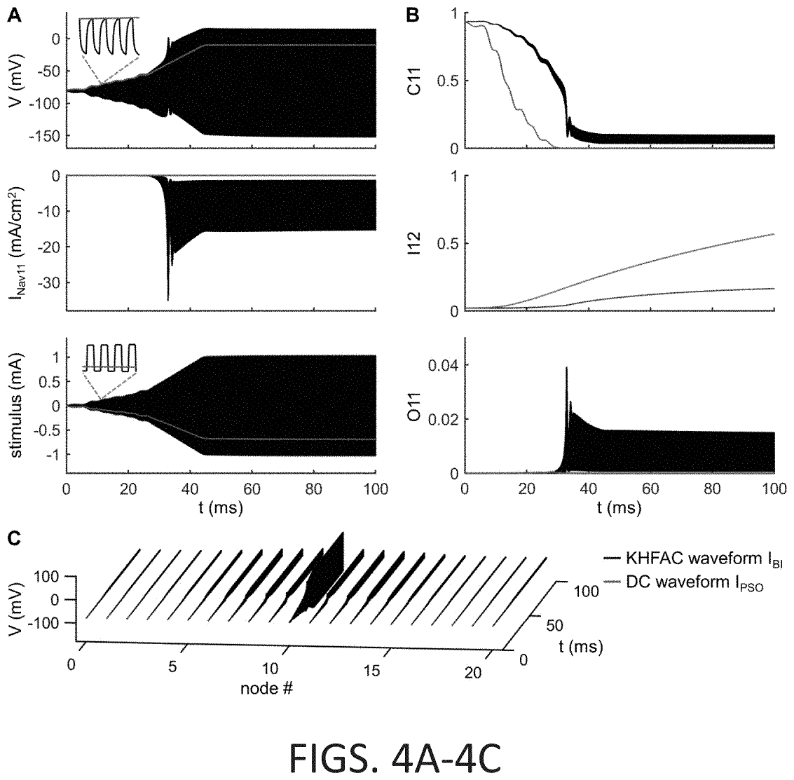

In some embodiments, the waveform that blocks neural conduction without inducing an onset response is an AC blocking waveform. In some embodiments, the AC blocking waveform comprises an amplitude envelope defined by the least one DC blocking waveform (see, e.g., FIG. 4).

In some embodiments, the optimized transmembrane voltage trajectory is identified using a computation model comprising characteristics of a nerve fiber. In some embodiments, these characteristics include, but are not limited to, the number of nodes of Ranvier, nerve fiber diameter, nerve fiber length, nerve fiber location, types of sodium channels, number of sodium channels, types of potassium channels, number of potassium channels, and degree of myelination. Other characteristics can also be included in the computational model, as would be recognized by one of ordinary skill in the art based on the present disclosure.

In some embodiments, the optimized transmembrane voltage trajectory is identified using a global optimization algorithm. In some embodiments, the global optimization algorithm includes, but is not limited to, a genetic algorithm, a particle swarm algorithm, a simulated annealing algorithm, an ant colony algorithm, an estimation of distribution algorithm, and any combinations and derivations thereof. In some embodiments, the optimized transmembrane voltage trajectory increases nonlinearly from a resting potential to a suprathreshold level, as described further herein.

In some embodiments, after a voltage trajectory is identified, the optimization algorithm is used to generate a plurality of candidate waveforms for driving the optimized voltage trajectory in an axon. In some embodiments, the optimization algorithm (e.g., PSO) is again applied, with the particles defined as the electrode currents at each time step, to design an extracellular stimulation waveform to drive PSO-generated voltage profile. The purpose of the PSO is to minimize the difference between the membrane voltage recorded in a particular node and the optimized voltage profile, as described further herein. As would be recognized by one of skill in the art based on the present disclosure, the optimized transmembrane voltage trajectory can also be identified using a global optimization algorithm. In some embodiments, the global optimization algorithm used to identify the at least one DC blocking waveform includes, but is not limited to, a genetic algorithm, a particle swarm algorithm, a simulated annealing algorithm, an ant colony algorithm, an estimation of distribution algorithm, and any combinations and derivations thereof.

In some embodiments, the AC blocking waveform can be applied at increasing amplitude over time (see, e.g., 4), which in part facilitates the elimination of the onset response. In some embodiments, the AC blocking waveform can be applied at increasing amplitude over a time period of 5 ms, 10 ms, 15 ms, 20 ms, 25 ms, 30 ms, 35 ms, 40 ms, 45 ms, 50 ms, 55 ms, 60 ms, 70 ms, 80 ms, 90 ms or 100 ms. In some embodiments, the AC blocking waveform can be applied at increasing amplitude over any time period after the initial application of the waveform (time 0) up until about 100 ms.

In some embodiments, the AC blocking waveform can be charge-balanced. In some embodiments, the AC blocking waveform can be biphasic, monophasic, or multiphasic. In other embodiments, the AC waveform can be multiphasic, including a waveform that is charge-balanced and/or biphasic. In some embodiments, the AC blocking waveform is symmetric and/or rectangular. In some embodiments, the AC blocking waveform can be sinusoidal. In some embodiments, the AC blocking waveform is a sawtooth. In some embodiments, the AC blocking waveform is triangular.

In some embodiments, the AC blocking waveform can be applied at a frequency of at least about 5 kHz. In some embodiments, the AC blocking waveform can be applied at a frequency of at least about 10 kHz. In some embodiments, the AC blocking waveform can be applied at a frequency of at least about 20 kHz. In some embodiments, the AC blocking waveform can be applied at a frequency of at least about 40 kHz. In some embodiments, the AC blocking waveform can be applied at a frequency of at least about 60 kHz. In some embodiments, the AC blocking waveform can be applied at a frequency of at least about 80 kHz. In some embodiments, the AC blocking waveform can be applied at a frequency of at least about 90 kHz. In some embodiments, the AC blocking waveform can be applied at a frequency of about 5 kHz to about 90 kHz. In some embodiments, the AC blocking waveform can be applied at a frequency of about 5 kHz to about 80 kHz. In some embodiments, the AC blocking waveform can be applied at a frequency of about 5 kHz to about 70 kHz. In some embodiments, the AC blocking waveform can be applied at a frequency of about 5 kHz to about 60 kHz. In some embodiments, the AC blocking waveform can be applied at a frequency of about 5 kHz to about 50 kHz. In some embodiments, the AC blocking waveform can be applied at a frequency of about 5 kHz to about 40 kHz. In some embodiments, the AC blocking waveform can be applied at a frequency of about 5 kHz to about 30 kHz. In some embodiments, the AC blocking waveform can be applied at a frequency of about 5 kHz to about 20 kHz. In some embodiments, the AC blocking waveform can be applied at a frequency of about 5 kHz to about 10 kHz. In some embodiments, the AC blocking waveform can be applied at a frequency of about 10 kHz to about 90 kHz. In some embodiments, the AC blocking waveform can be applied at a frequency of about 20 kHz to about 90 kHz. In some embodiments, the AC blocking waveform can be applied at a frequency of about 30 kHz to about 90 kHz. In some embodiments, the AC blocking waveform can be applied at a frequency of about 40 kHz to about 90 kHz. In some embodiments, the AC blocking waveform can be applied at a frequency of about 50 kHz to about 90 kHz. In some embodiments, the AC blocking waveform can be applied at a frequency of about 60 kHz to about 90 kHz. In some embodiments, the AC blocking waveform can be applied at a frequency of about 70 kHz to about 90 kHz. In some embodiments, the AC blocking waveform can be applied at a frequency of about 80 kHz to about 90 kHz.

### 3. NEUROMODULATION SYSTEMS

Embodiments of the present disclosure also include the various AC conduction block waveforms described herein, as well as systems for blocking neural conduction without inducing an onset response in a subject using the AC blocking waveforms. In accordance with these embodiments, the AC blocking waveform can applied at increasing amplitude over time, which blocks neural conduction and induces closed-state inactivation (CSI) of voltage-gated sodium channels prior to activation. The application of the AC blocking waveform using these systems eliminates onset firing that often causes muscle contraction and pain in a subject, which is a significant impediment to potential clinical applications of KHFAC nerve block.

In some embodiments, the system includes an electrode sized and configured for implantation in proximity to neural tissue, and a pulse generator coupled to the electrode. In some embodiments, the pulse generator includes a power source comprising a battery and a microprocessor coupled to the battery. In some embodiments, the pulse generator is capable of applying to the electrode an AC waveform capable of blocking neural conduction and eliminating onset response by inducing closed-state inactivation (CSI) of voltage-gated sodium channels prior to activation.

In some embodiments, the AC blocking waveform is applied at increasing amplitude over time, as described further herein. In some embodiments, the AC blocking waveform is charge-balanced. In some embodiments, the AC blocking waveform is biphasic, monophasic, or multiphasic. In some embodiments, the AC blocking waveform is symmetric and/or rectangular. In some embodiments, the AC blocking waveform is applied at a frequency of about 5 kHz to about 90 kHz, as described further herein.

Embodiments of the present disclosure also include a method for blocking neural conduction in a subject using any of the systems described herein. In accordance with these embodiments, the method includes programming the pulse generator to output the blocking waveform at increasing amplitude over time, and delivering the blocking waveform to the subject without inducing an onset response.

Embodiments of the present disclosure also include methods for blocking neural conduction without inducing onset response using embodiments of the systems and methods as described above. In accordance with these embodiments, the methods include programming the pulse generator to output the waveform (e.g., on a graphical user interface (GUI)). In some embodiments, the waveform is an AC blocking waveform with an amplitude envelope that is defined by a DC stimulation waveform. In some embodiments, the AC waveform blocks neural conduction without inducing an onset response when delivered by the pulse generator.

In some embodiments, the electrode or lead can be placed in a desired position in contact with nervous system tissue of a subject receiving neural block conduction treatment. In one embodiment, the electrode can be implanted in a region of the brain, such as the thalamus, subthalamus, subthalamic nucleus, or globus pallidus for the purpose of deep brain stimulation. However, as would be recognized by one of ordinary skill in the art based on the present disclosure, the electrode can be implanted in, on, or near the spinal cord; or in, on, or near a peripheral nerve (sensory or motor or mixed; somatic or autonomic); or in, or, or near a neural plexus; or in, on, or near any subcutaneous tissue such as muscle tissue (including cardiac tissue) or adipose tissue or other organ tissue for the purpose of stimulation to achieve a therapeutic purpose. In addition, the electrode may be utilized for transcutaneous stimulation where electrodes are placed, not subcutaneous, but on an outer skin surface. Further, the electrode may be utilized for percutaneous stimulation where electrodes are placed through the skin but the pulse generator remains outside the skin.

The electrode can be one or more electrodes configured as part of the distal end of a lead or be one or more electrodes configured as part of a leadless system to apply electrical pulses to the targeted tissue region. Electrical pulses can be supplied by a pulse generator coupled to the electrode/lead. In one embodiment, the pulse generator can be implanted in a suitable location remote from the electrode/lead (e.g., in the shoulder region); however, that the pulse generator could be placed in other regions of the body or externally to the body.

When implanted, at least a portion of the case or housing of the pulse generator can serve as a reference or return electrode. Alternatively, the lead can include a reference or return electrode (comprising a multipolar (such as bipolar) arrangement), or a separate reference or return electrode can be implanted or attached elsewhere on the body (comprising a monopolar arrangement).

The pulse generator can include stimulation generation circuitry, which can include an on-board, programmable microprocessor, which has access to and/or carries embedded code. The code expresses pre-programmed rules or algorithms under which desired electrical stimulation is generated, having desirable electrical stimulation parameters that may also be calculated by the microprocessor, and distributed to the electrode(s) on the lead. According to these programmed rules, the pulse generator directs the stimulation through the lead to the electrode(s), which serve to selectively stimulate the targeted tissue region. The code may be programmed, altered or selected by a clinician to achieve the particular physiologic response desired. Additionally or alternatively to the microprocessor, stimulation generation circuitry may include discrete electrical components operative to generate electrical stimulation having desirable stimulation parameters, as described further herein (e.g., input to generate an optimized waveform shape, which can include a pulse amplitude; a pulse width (PW) or duration; a frequency of stimulation pulses applied over time; and a shape or waveform of the stimulation pulses). One or more of the parameters may be prescribed or predetermined as associated with a particular treatment regime or indication. In some embodiments, the pulse generator can be programed to output a stimulation waveform (e.g., on a graphical user interface (GUI)), and the stimulation waveform can represent a waveform having an optimized shape capable of blocking neural conduction without inducing onset response, as described further herein. In some embodiments, programming the pulse generator includes setting the amplitude of the stimulation waveform, such that the stimulation waveform blocks neural conduction when delivered by the pulse generator.

Additionally, the neuromodulation systems and methods of the present disclosure include a system for delivering neuromodulation therapy to a subject in order to reduce, treat, or prevent the subject's neuropathic pain. In accordance with these embodiments, the system includes an electrode sized and configured for implantation in proximity to neural tissue. For example, the system can include a stimulation device, an electrical connection lead, and at least one electrode or electrode array operatively positioned in the epidural space of a vertebral column of a subject that is experiencing neuropathic pain. The electrode or electrode array can be positioned at the site of nerves that are the target of stimulation (e.g., along the spinal cord), or positioned in any suitable location that allows for the delivery of electrical stimulation to the targeted neural tissue.

In some embodiments, the system includes a pulse generator coupled to the electrode. The pulse generator can include a power source comprising a battery and a microprocessor coupled to the battery, and the pulse generator is generally configured to generate electrical signals for delivering neuromodulation therapy. In some embodiments, the system further includes a controller comprising hardware, software, firmware, or combinations thereof for implementing functionality described herein. For example, the controller can be implemented by one or more processors and memory. The controller can be operatively connected to the pulse generator to facilitate the generation of electrical signals and applying temporal patterns of electrical stimulation to targeted neurological tissue. The output signals may be received by the connection lead and carried to the electrode or electrode array for the delivery of electrical stimulation to targeted neurological tissue. The system can include a power source, such as a battery, for supplying power to the controller and the pulse generator.

In some embodiments, the system also includes an external computing device that is not implanted within the subject. The computing device can communicate with a stimulation device or system via any suitable communication link (e.g., a wired, wireless, or optical communication link). The communication link may also facility battery recharge. A clinician may interact with a user interface of the computing device for programming the output of the implanted pulse generator, including the electrodes that are active, the stimulation pulse amplitude, the stimulation pulse duration, the stimulation pattern (including pulse repetition frequency), and the like applied via each electrode contact to each sub-population. In accordance with these embodiments, systems and methods of the present disclosure can be used to deliver an AC blocking waveform, as described herein, to reduce pain in a plurality of subjects with different pain states without inducing onset response.

In some embodiments, systems and methods of the present disclosure can be implemented as an algorithm within a pulse generator device. An on-board controller can deliver multiple frequencies and patterns through different output channels to different contacts on an electrode, including delivering any of the AC blocking waveforms of the present disclosure. Values of the stimulation frequencies and patterns of stimulation and the electrodes through which these frequencies and patterns are delivered can be input by either a physician or a patient through a user interface. Alternatively, the device can be pre-programmed with specific combinations of frequencies and patterns to use. The applied frequencies and patterns may or may not be offset from each other at the start of stimulation. The algorithm can be toggled on and off (e.g., between multi-frequency and single frequency) by either the physician or patient, or it can be coupled to an internal feedback-driven algorithm for automatic control.

### 4. MATERIALS AND METHODS

Axon Model.

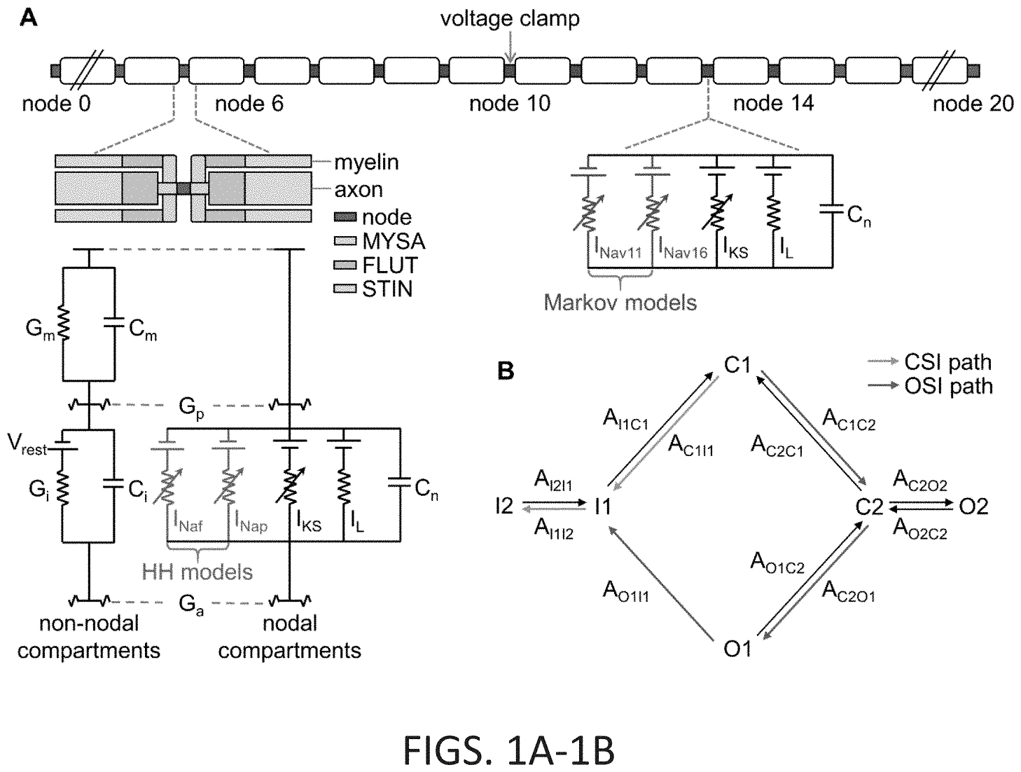

The MRG double-cable myelinated axon model was used to simulate KHFAC conduction block, and this model was originally developed to reproduce a wide range of excitation properties of mammalian peripheral nerve fibers. The model was composed of equivalent electrical circuits for both nodal and internodal segments (FIG. 1A). The nodal circuit included fast Na⁺ (I_{Naf}), persistent Na⁺ (I_{Nap}), slow K⁺ (I_{KS}), and linear leakage (I_{L}) currents, and the membrane capacitance Cₙ, and the ionic currents were originally modeled by using HH-type kinetics. I_{Naf} was responsible for AP initiation in each node (maximum conductance: g̅_{Naf}=3 S/cm²), and I_{Nap} was responsible for generating a non-inactivating current at the afterpotentials (maximum conductance: g̅_{Nap}=0.01 S/cm²). The resting potential in each node was −80 mV. The axon-specific parameters were implemented following McIntyre et al. (Tables 1 and 2) and were previously validated to be consistent with experimental observations of KHFAC conduction block. The axon models were solved in the NEURON simulation environment (version 7.7) using a time step of 0.005 ms.

Markov-Type Models of VGSCs.

There are two types of models commonly used to reproduce the electrophysiological behaviors of VGSCs, i.e., HH- and Markov-type models. The HH-type models represent an ionic channel as an assembly of several independent gating particles, which are not sufficient to capture specific kinetics of VGSCs. Unlike the HH formalism, Markov-type models represent an ion channel as a series of conformational states of the channel protein and a collection of transitions between them, which allowed for reproducing the dependence of Na⁺ inactivation on activation. Markov-type kinetic models of VGSCs were used to design the transmembrane voltage trajectory for causing Na⁺ CSI.

The Markov-type models used in the simulations were developed by Balbi et al. and were validated against electrophysiological data from all VGSC isoforms. The model framework was detailed, unifying, and computationally efficient, and can account for different features of each human VGSC isoform with a minimal set of states and transitions. The model included six states, and the state diagram and transitions of the Markov-type channels are shown in FIG. 1B. C1 and C2 were two closed states, O1 and O2 were two open states, and I1 and I2 were two inactivation states. The transition from O1 to I1 was irreversible, and the transitions between the other consecutive states were reversible. The dynamics of fraction of VGSCs in each state were governed by following Markovian equations

dC1/dt=A_{I1C1}I1+A_{C2C1}C2−(A_{C1C2}+A_{C1I1})C1 (1)

dC2/dt=A_{C1C2}C1+A_{O1C2}O1+A_{O2C2}O2−(A_{C2C1}+A_{C2O1}+A_{C2O2})C2 (2)

dO1/dt=A_{C2O1}C2+A_{I1O1}I1−(A_{O1C2}+A_{O1I1})O1 (3)

dO2/dt=A_{C2O2}C2−A_{O2C2}O2 (4)

dI1/dt=A_{I2I1}I2+A_{C1I1}C1+A_{O1I1}O1−(A_{I1C1}+A_{I1I2}+A_{I1O1})I1 (5)

dI2/dt=A_{I1I2}I1−A_{I2I1}I2 (6)

Here A_{w1w2} was the transition rate from state w1 to state w2, which was computed by

$${A}_{w 1 w 2} = {{B}_{hyp}^{w 1 w 2} [ 1 + exp ( \frac{V - {V}_{hyp}^{w 1 w 2}}{{k}_{hyp}^{w 1 w 2}} ) ]}^{- 1} + {{B}_{dep}^{w 1 w 2} [ 1 + exp ( \frac{V - {V}_{dep}^{w 1 w 2}}{{k}_{dep}^{w 1 w 2}} ) ]}^{- 1} \qquad ( 7 )$$

The Na⁺ conductance was determined by the fraction of open channels, and the Na⁺ current was computed by

I_{Nav}=g̅_{Nav}(O1+O2)(V−E_{Na}) (8)

where g̅_{Nav} was the maximum conductance, V was local membrane voltage, and E_{Na}=50 mV was the Na⁺ reversal potential.

Implementation of Markov-Type VGSCs.

KHFAC waveforms are applied in practice using comparatively small electrodes and generate a locally-acting conduction block in peripheral nerves. In the simulations, there were 21 nodes in the model axon and the simulated block electrode was placed above the central node (i.e. node 10). To reduce overall computational demands, Markov-type VGSCs were implemented in the central nine nodes, which were closest to the block electrode. The other nodes far away from the site of block were modeled with HH-type VGSCs. Note that the specific number of Markov-type nodes did not alter the block threshold or lack of onset response in the hybrid model (FIG. 11).

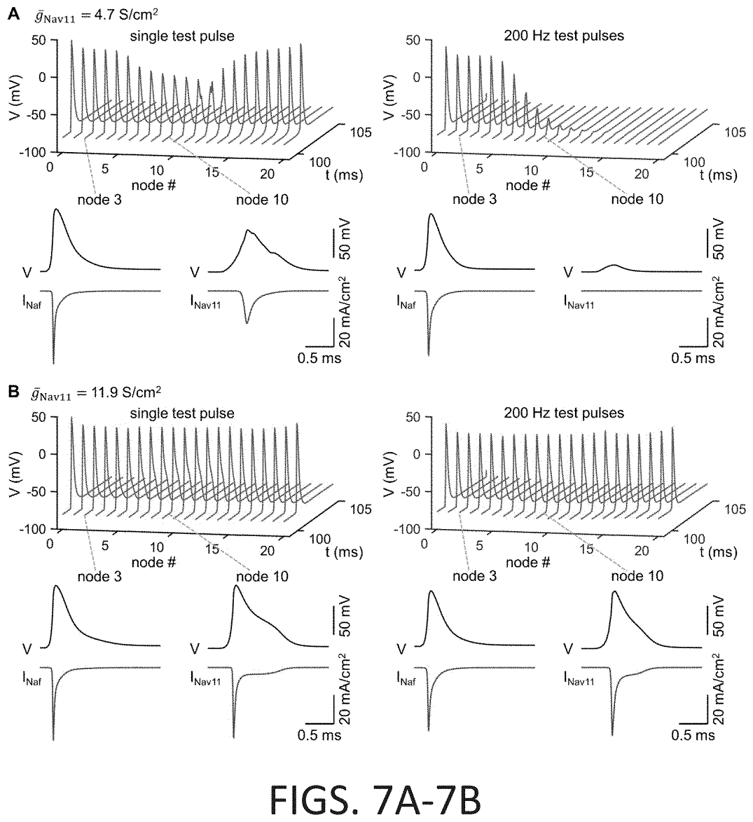

Experimental data indicated that the Naᵥ 1.1 and Naᵥ 1.6 channels are preferentially expressed in the nodes of mammalian peripheral nerve fibers. The HH-type models of/Naf and I_{Nap} were substituted in node 6 to node 14 with the Markov-type models of Naᵥ 1.1 and Naᵥ 1.6 channels, respectively. The parameters of each transition rate for two VGSCs are provided in Table 3. Only the maximum conductances of I_{Nav11} and I_{Nav16} were modified, and this did not change any parameter of their transition rates between consecutive states. The maximum conductance of Naᵥ 1.6 channels was set to a g̅_{Nav16}=0.01 S/cm², which was identical to that of I_{Nap}. For Naᵥ 1.1 channels, if g̅_{Nav11} was set to the minimal value (4.7 S/cm²) for faithfully propagating a single spike from node 0 to node 20, there was substantial attenuation in the AP as it propagated from HH-type nodes to Markov-type nodes (FIG. 7). In this case, the g̅_{Nav11} was too small to propagate APs at high frequencies. For example, when firing rate was 200 spikes per second, APs were lost as they propagated along the axon. g̅_{Nav11} was set to the minimal value (11.9 S/cm²) to propagate faithfully spike trains at rates of up to 400 spikes per second. At a g̅_{Nav11}=11.9 S/cm², there was no attenuation in the APs as they propagated along the axon (FIG. 7). Although a g̅_{Nav11} was larger than g̅_{Naf}, I_{Nav11} and I_{Naf} had similar magnitude underlying an AP.

Particle Swarm Optimization.

PSO algorithms were applied to design the transmembrane voltage trajectory for causing Na⁺ CSI and to generate current waveform to drive this voltage trajectory, which were implemented in Matlab (version R2016a). PSO is a meta-heuristic optimization method inspired by the information circulation and social behavior of swarms. It uses a population-based search strategy to solve nonlinear optimization problems, based on cooperation and competition among the particles of a swarm. In PSO, each particle is a point in the search space, which represents a candidate solution of the optimization problem. The movement of a particle in the search space is driven by its own fitness as well as the highest fitness in the swarm. The algorithm iteratively updates the velocity of each particle towards the position of the highest fitness. Through communication between particles over multiple iterations, the particles explore the problem space to identify the optimal solution of the problem.

Mathematically, the position of particle i (1≤i≤M) at the kth iteration was described by a vector \vec{X}ᵢ(k)=[x_{i,1}(k), x_{i,2}(k), . . . , x_{i,D}(k)], where M was the number of particles and D was the dimension of the search space. Each dimension of the particle was bounded between xₘᵢₙ and xₘₐₓ. The personal best position previously found by particle i was \vec{P}ᵢ=[p_{i,1}, p_{i,2}, . . . , p_{i,D}], and the best position experienced by all particles in the whole swarm was indicated by \vec{G}=[g₁, g₂, . . . , g_{D}]. The velocity of particle i in the kth iteration was \vec{V}ᵢ(t)=[v_{i,1}(k), v_{i,2}(k), . . . , v_{i,D}(k)], which determined the movement direction of the particle through the search space. At the kth iteration, the d-dimension (1≤d≤D) of particle i was updated based on following equations

v_{i,d}(k+1)=c{r₁[p_{i,d}−x_{i,d}(k)]+r₂[g_{d}−x_{i,d}(k)]} (9)

x_{i,d}(k+1)=x_{i,d}(k)+v_{i,d}(k+1) (10)

where c=1.5 was a learning coefficient, and r₁ and r₂ were uniform random numbers within [0, 1] used to help promote exploration.

Implementation of PSO.

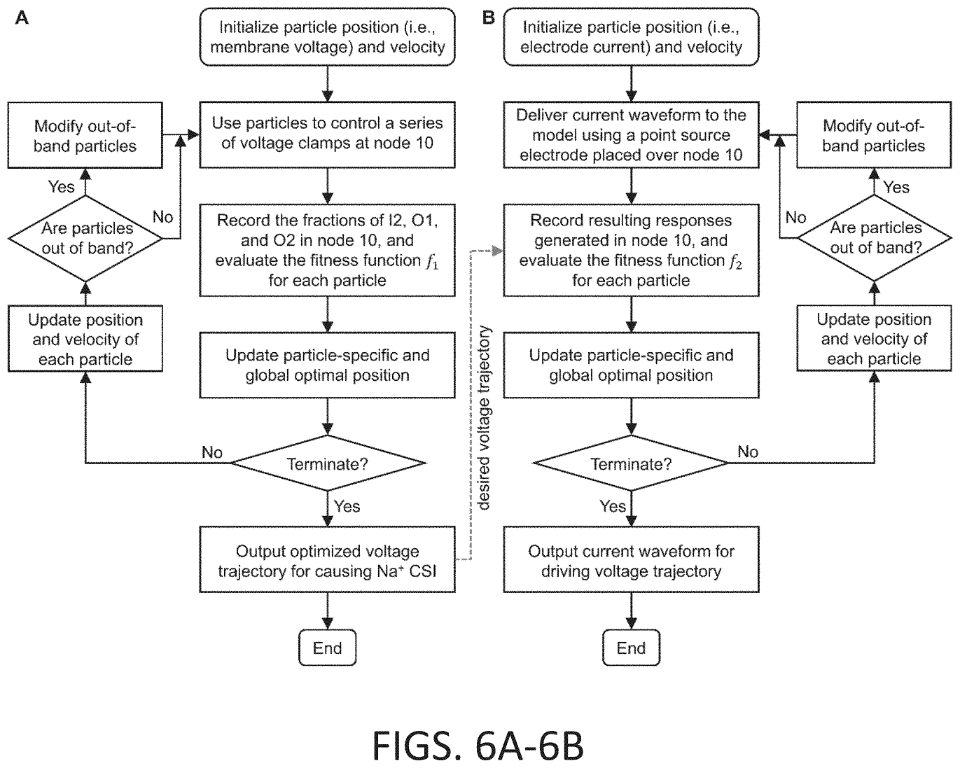

PSO was applied to generate an optimized voltage trajectory V_{PSO} for causing the CSI of Naᵥ 1.1 and Naᵥ 1.6 channels in the 21-node model (FIG. 6A). A population of 50 particles was defined as the membrane voltages used to control a time series of voltage clamps at node 10. The Na⁺ currents were recorded through Naᵥ 1.1 and Naᵥ 1.6 channels as well as their fractions in states C1, I1, 12, and O1 during voltage clamp. A multi-objective optimization problem was defined: find a voltage trajectory to maximize the fractions of Naᵥ 1.1 and Naᵥ 1.6 channels in inactivated states while minimizing the fractions of the two VGSCs in open states. Mathematically, the following cost function ƒᵢ was defined to evaluate each solution,

Minimize ƒₜ=Σⱼ₌₁^{N}[B(O11ⱼ+O12ⱼ+O61ⱼ+O62ⱼ)−(I12ⱼ+I62ⱼ)] (11)

where O11, O12, and I12 were the fractions of Naᵥ 1.1 channels in states O1, O2 and I2, O61, O62, and I62 were the fractions of Naᵥ 1.6 channels in states O1, O2 and 12, j corresponded to the time point, and B=20000 was a weight used to reduce the fractions in open states. The lower bound of each particle was xₘᵢₙ=−80 mV (i.e., resting potential), and the upper bound was xₘₐₓ=−10 mV. The time constant of inactivation state I1 was smaller than I2, and the optimization results were sensitive to the changes in I1. Thus, the fraction of state I1 was excluded from the fitness function ƒ₁. The initial positions of 50 particles were defined to be randomly distributed around a straight line, which linearly increased from xₘᵢₙ to xₘₐₓ.

Subsequently, PSO was used to generate a current waveform for driving the optimized voltage trajectory in the central node of the 21-node model (FIG. 6B). A point source electrode was placed over node 10 at an electrode-fiber distance of 1 mm. Fifty particles were defined as a time series of electrode currents. The extracellular medium was assumed to be infinite homogenous and isotropic with a conductivity of 6=0.303 S/m. For a segment at location (x, y, z), the extracellular potential Vₑₓₜ was calculated as

$${V}_{e x t} ( x , y , z ) = \frac{{I}_{e x t}}{4 π σ \sqrt{{( x - {x}_{0} )}^{2} + {( y - {y}_{0} )}^{2} + {( z - {z}_{0} )}^{2}}} \qquad ( 12 )$$

where Iₑₓₜ was the amplitude of electrode current, and (x₀, y₀, z₀) was the location of the point source electrode. The resulting transmembrane voltage was recorded in node 10 at each iteration. V_{PSO} was set as the desired voltage trajectory, and defined an one-objective optimization problem: find electrode currents to minimize the difference between transmembrane voltage V_{node 10} recorded in node 10 and desired voltage trajectory V_{PSO}. Mathematically, the following cost function ƒ₂ was defined to evaluate the quality of this problem solution

Minimize ƒ₂=Σⱼ₌₁^{N}(v_{node 10,j}−V_{PSO,j})² (13)

where j corresponded to the time point. By using the point source electrode, cathodic stimulus depolarized node 10 and anodic stimulus hyperpolarized node 10. Since V_{PSO} started from the resting potential, the upper bound of each particle was xₘₐₓ=0 mA. The lower bound was defined to xₘᵢₙ=−0.7 mA, which was sufficient to drive transmembrane voltage in node 10 to a plateau level higher than −10 mV. The initial positions of particles were defined to be randomly distributed around a straight line from the xₘᵢₙ to xₘₐₓ.

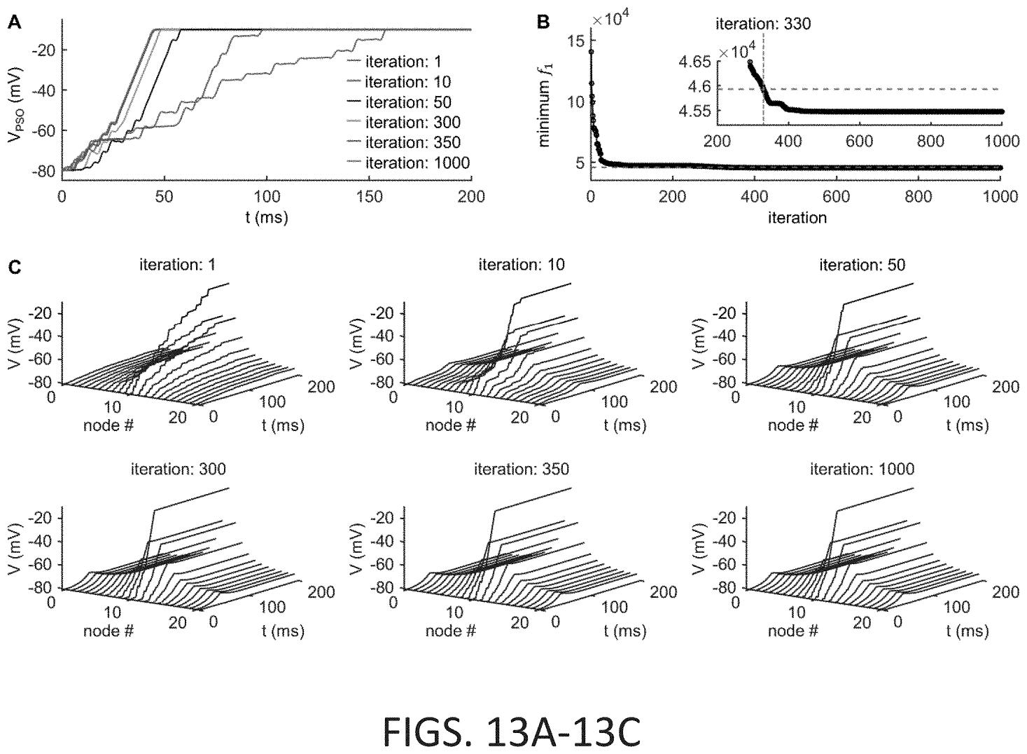

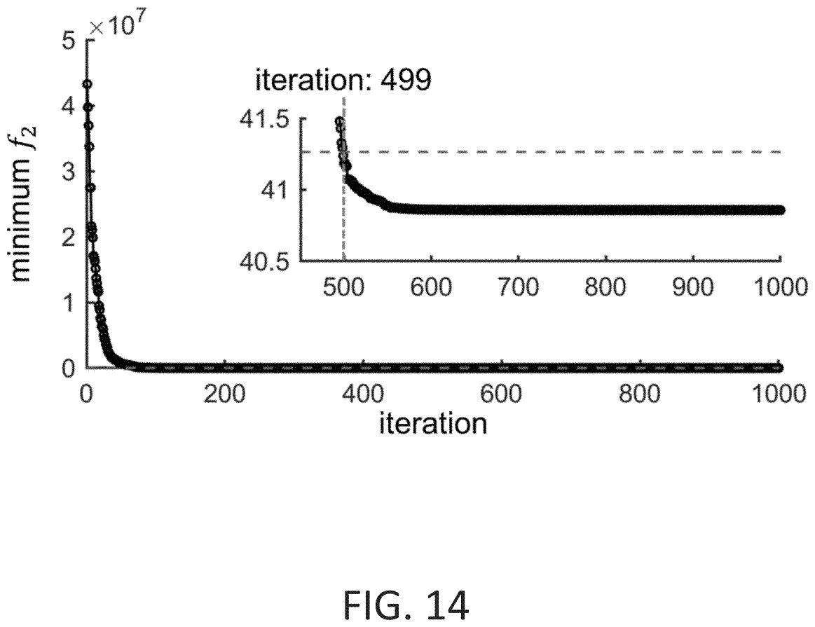

When solving the optimal solutions for ƒₜ and ƒ₂, the time step of PSO was 2 ms and the search space dimension was D=50. The termination criterion was that the algorithm generation reached a limit of 1000, which was sufficient for convergence. When designing the optimized voltage trajectory V_{PSO} for causing Na⁺ CSI, the minimum ƒᵢ converged to within 1% of the final iteration by 330 iterations (FIG. 13). When generating the DC waveform I_{PSO} for driving the optimized trajectory V_{PSO}, the minimum fitness ƒ₂ after 499 iterations converged to within 1% of the final iteration (FIG. 14).

Simulation Protocols for KHFAC Conduction Block.

After generating PSO-based DC and KHFAC waveforms for conduction block, experiments were conducted to examine whether they activated onset responses. An extracellular point source electrode was used to deliver blocking currents to the central node (10) of the model, and the electrode-fiber distance was 1 mm. Subsequently, it was determined whether the PSO-generated waveform blocked nerve conduction. An intracellular monopolar electrode was applied to deliver suprathreshold test pulses (width: 0.1 ms and amplitude: 2.5Iₜₕ) at one end of the axon (node 0), which was used to stimulate APs in node 0 after the onset of blocking currents and test whether these APs propagated through the site of the block electrode. Iₜₕ=0.43 mA was the threshold of intracellular test pulse for activation of an AP in node 0.

A 251-node model was used to examine the conduction block at the fiber diameters (2 μm and 8.7 μm) less than 10 μm using PSO-based KHFAC waveform. Each fiber diameter had specific lengths for all segments (Table 1), which were identified based on experimental data. The Markov-type Naᵥ 1.1 and Naᵥ 1.6 channels were implemented in node 121 to node 129, and the block electrode was placed 1 mm above node 125.

### 5. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Design of Transmembrane Voltage Trajectory to Generate Closed-State Inactivation of VGSCs.

The first step was to design a voltage trajectory that generated CSI of VGSCs. A 21-node 10 μm diameter MRG myelinated axon model of mammalian peripheral nerve fiber was used (FIG. 1A). The fast Na⁺ (I_{Naf}) and persistent Na⁺ (I_{Nap}) currents in each node were originally modeled using Hodgkin-Huxley (HH)-type channels, which represented a VGSC as an assembly of several independent gating variables and were unable to represent the dependence of Na⁺ inactivation on activation. Since KHFAC waveforms generate a locally-acting conduction block in peripheral nerves, the original I_{Naf} and I_{Nap} were replaced respectively by Markov-type Naᵥ 1.1 and Naᵥ 1.6 channels in the middle nine nodes (6 to 14) to create a hybrid model that enabled transitions of VGSCs between states. The CSI path of Naᵥ 1.1 and Naᵥ 1.6 channels was C1→I1→I2, and the OSI path was C1→C2→O1→I1→I2 (FIG. 1B). The original conductance density of I_{Naf} was too small for I_{Nav11} to propagate faithfully an AP from node 0 to node 20, and the conductance density of I_{Nav11} was increased to enable high fidelity propagation along the hybrid axon model at rates of up to 400 spikes per second (FIG. 7).

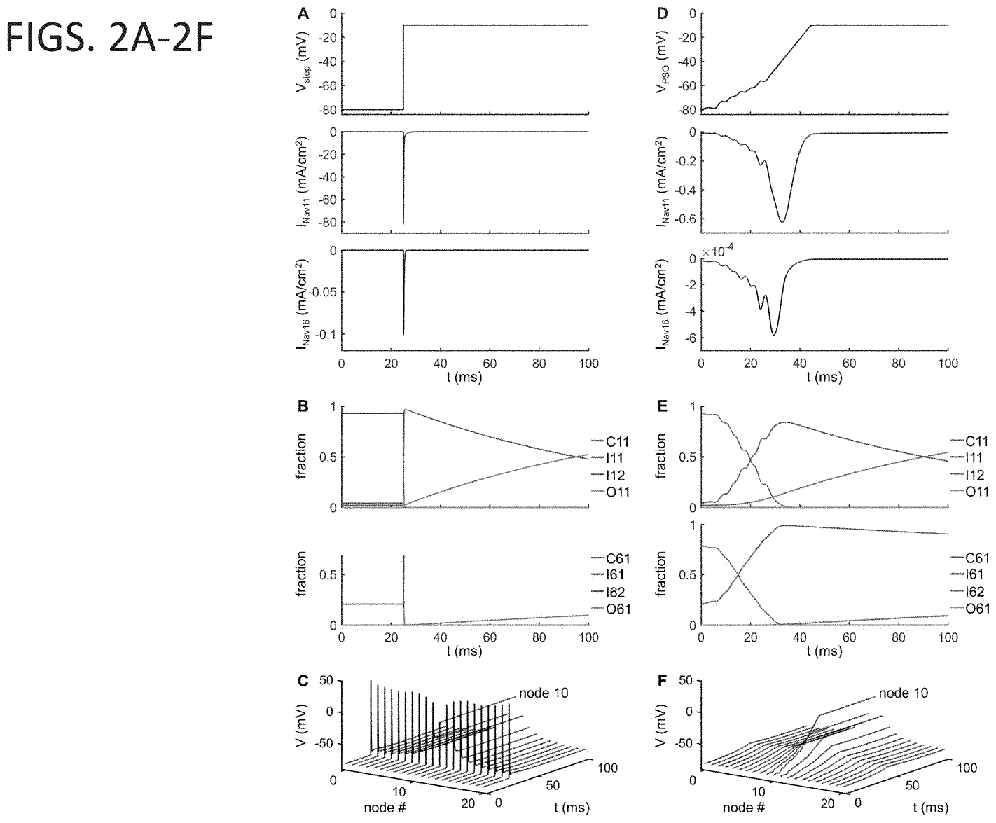

The responses in Naᵥ 1.1 and Naᵥ 1.6 channels were first examined when a standard voltage step (from −80 mV to −10 mV) was applied to the central node as a voltage clamp (FIG. 2A). At the resting potential of −80 mV, the two VGSCs were in closed states C1. After the onset of the voltage step to −10 mV, both Naᵥ 1.1 and Naᵥ 1.6 channels first entered open states O1 and then inactivated (FIG. 2B), i.e., OSI occurred. The magnitude of I_{Nav11} and I_{Nav16} reached −81.85 mA/cm² and −0.10 mA/cm², respectively. The conductance density of Naᵥ 1.6 channels (i.e., 0.01 mS/cm²) was much smaller than that of Naᵥ 1.1 channels (i.e., 11.9 mS/cm²), and I_{Nav11} was much larger than I_{Nav16}. An AP was initiated and propagated in the hybrid model axon at the onset of voltage step (FIG. 2C).

Subsequently, PSO was applied to design an optimized trajectory of transmembrane voltage to drive transitions along the CSI path. PSO is a search algorithm inspired by the social behavior of swarms. It finds the optimal or near-optimal solutions to numerical and qualitative problems by adjusting the trajectories of individual particles in the defined search space of an objective function, and is particularly applicable to addressing waveform optimization. A population of 50 particles was designed as the membrane voltages that were used as the voltage profile to control a series of voltage clamps at node 10. The objective of the PSO was to maximize the fractions of Naᵥ 1.1 and Naᵥ 1.6 channels in the inactivated states while minimizing the fractions in the open states. The PSO-generated voltage profile V_{PSO} increased nonlinearly from the resting potential (−80 mV) to a suprathreshold level (−10 mV) over a duration of 46 ms (FIG. 2D, top) and drove transitions of Naᵥ 1.1 and Naᵥ 1.6 channels to CSI.

The transitions from the closed states to the inactivated states caused by voltage profile V_{PSO} were similar for Naᵥ 1.1 and Naᵥ 1.6 channels. At the resting potential, VGSCs were in C1 state. After depolarization, the fraction of either channel in C1 was gradually reduced (FIG. 2E), the fractions in I2 increased, and there was little increase in the fraction in O1. Thus, more Naᵥ 1.1 and Naᵥ 1.6 channels were driven to inactivation directly from the resting state, which reduced the availability of each channel to conduct and participate in AP generation, and when the membrane voltage reached suprathreshold levels, the VGSCs were unable to enter the open states. The Na⁺ conductance was determined by the fraction of VGSCs in the open states, and since few Naᵥ 1.1 channels entered the O1 state during V_{PSO}, I_{Nav11} reached only −0.62 mA/cm² (FIG. 2D, bottom). In contrast to the voltage clamp step, the PSO-generated V_{PSO} successfully moved both Naᵥ 1.1 and Naᵥ 1.6 channels to CSI, and V_{PSO} delivered as a series of voltage clamps to the central node did not evoke an AP (FIG. 2F).

### Example 2

PSO-Generated DC Waveform Drives V_{PSO} and Generates Onset-Free Conduction Block.

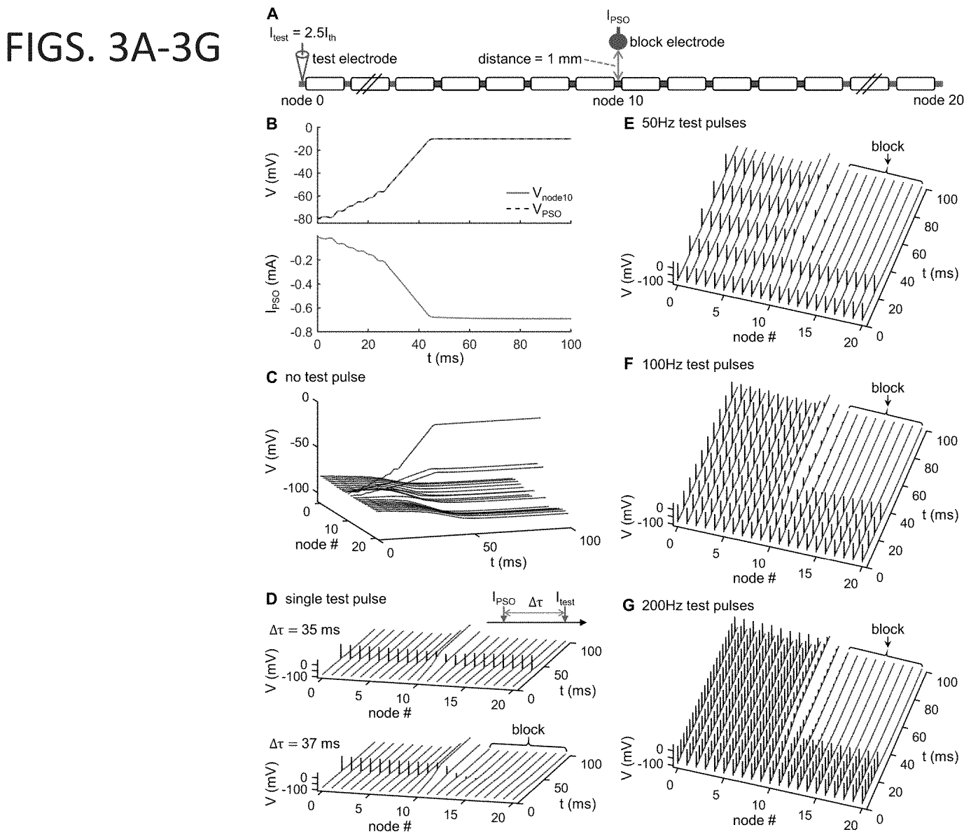

Having generated a transmembrane voltage trajectory that caused CSI of Naᵥ 1.1 and Naᵥ 1.6 channels, the next step was to design a stimulus waveform to drive this voltage profile in the axon. PSO was again applied, with the particles defined as the electrode currents at each time step, to design an extracellular stimulus to drive V_{PSO} in node 10 using an extracellular monopolar electrode placed 1 mm above the central node (FIG. 3A). The goal of the PSO was to minimize the difference between the membrane voltage recorded in node 10 and the optimized voltage profile V_{PSO}. The resulting current I_{PSO} increased nonlinearly from 0 mA to a plateau of −0.69 mA (FIG. 3B) and drove the transmembrane potential to track V_{PSO}. No AP was generated in the axon by I_{PSO} (FIG. 3C) and it successfully blocked nerve conduction. When the time delay (Δτ) between the onset of an intracellular test pulse and the onset of I_{PSO} was ≤35 ms, the single spike propagated through the axon (FIG. 3D). With Δτ≥36 ms, the transmission of single spike was blocked (FIG. 3D), as were spike trains at 50 Hz, 100 Hz or 200 Hz (FIGS. 3E-3G). Thus, the PSO-generated waveform I_{PSO} blocked nerve conduction without activating an onset response in the hybrid model axon.

### Example 3

KHFAC Waveform for Onset-Free Conduction Block.

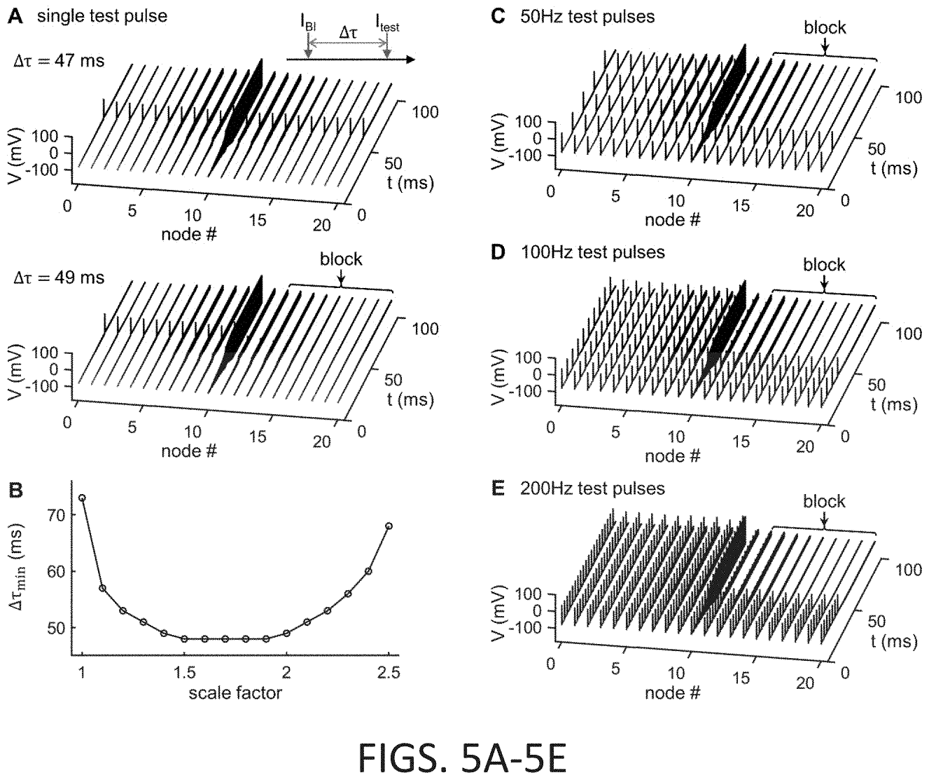

The DC I_{PSO} waveform was not charge-balanced, and thus can cause irreversible faradaic reactions at the electrode-electrolyte interface, and these may result in electrode and or nerve damage when applied for long periods of time. Therefore, a scaled version of the DC I_{PSO} waveform was used as the envelope of symmetric rectangular charge-balanced biphasic pulses, applied at 10 kHz to block nerve conduction (FIG. 4A, bottom). However, there was a time delay after the onset of resulting KHFAC waveform I_{BI} and complete conduction block (FIG. 5A), and this delay depended on the amplitude scale factor (FIG. 5B). The minimum scale factor for producing conduction block was 1.0, but a scale factor of 1.5 was used, which enabled the KHFAC waveform to generate conduction block after a minimum time delay.

The KHFAC waveform I_{BI}, delivered using a monopolar electrode placed 1 mm over node 10, generated passive oscillations in transmembrane voltage at node 10, and the upper envelope of the transmembrane voltage response followed the optimized voltage profile V_{PSO} (FIG. 4A, top). The intrinsic low-pass filtering and rectification of AC electrical signals by the cell membrane prevented neural firing from following the KHFAC stimulus, while the envelope of the PSO-based waveform I_{BI} drove the Naᵥ 1.1 channels to CSI (FIG. 4B). The fraction C11 of Naᵥ 1.1 channels in C1 state was higher than that in response to the DC waveform I_{PSO} (FIG. 4B), which enabled more channels to enter O1 state instead of I2 and resulted in more Na⁺ influx through the Naᵥ 1.1 channels, causing a depolarized peak in the transmembrane voltage of node 10. Importantly, this depolarized peak did not generate a propagating AP (FIG. 4C), and the KHFAC waveform enabled onset-free conduction block. When the time delay (Δτ) between the onset of an intracellular test pulse and the onset of I_{BI} was ≤47 ms, the single spike propagated through the axon (FIG. 5A). With Δτ≥48 ms, the transmission of the single spike was blocked (FIG. 5A), and the minimum delay Δτₘᵢₙ for producing conduction block was dependent on the scale factor (FIG. 5B), reaching a minimum when the scale factor was 1.5˜1.9. Further, I_{BI} completely blocked conduction of spike trains at 50 Hz, 100 Hz, or 200 Hz (FIGS. 5C-5E). Thus, the PSO-based charged-balanced KHFAC waveform blocked nerve conduction without generating an onset response.

Additional support for the various embodiments described herein can be found in the following tables.

TABLE 1
Geometric parameters of MRG models.

| Fiber Diameter | 2.0 μm [1] | | 8.7 μm [2] | | 10.0 μm [2] | |
|---|---|---|---|---|---|---|
| internodal length | 200 | μm | 1000 | μm | 1150 | μm |
| number of myelin | 30 | μm | 110 | μm | 120 | μm |
| lamella | | | | | | |
| node length | 1 | μm | 1 | μm | 1 | μm |
| node diameter | 1.4 | μm | 2.8 | μm | 3.3 | μm |
| MYSA length | 3 | μm | 3 | μm | 3 | μm |
| MYSA diameter | 1.4 | μm | 2.8 | μm | 3.3 | μm |
| MYSA periaxonal | 0.002 | μm | 0.002 | μm | 0.002 | μm |
| space width | | | | | | |
| FLUT length | 10 | μm | 40 | μm | 46 | μm |
| FLUT diameter | 1.6 | μm | 5.8 | μm | 6.9 | μm |
| FLUT periaxonal | 0.004 | μm | 0.004 | μm | 0.004 | μm |
| space width | | | | | | |
| STIN length | 57.7 | μm | 152.2 | μm | 175.2 | μm |
| STIN diameter | 1.6 | μm | 5.8 | μm | 6.9 | μm |
| STIN periaxonal | 0.004 | μm | 0.004 | μm | 0.004 | μm |
| space width | | | | | | |

[1] McIntyre CC, Grill WM, Sherman DL, Thakor NV. Cellular effects of deep brain stimulation: model-based analysis of activation and inhibition. J Neurophysiol. 2004 Apr; 91(4): 1457-69.
[2] McIntyre CC, Richardson AG, Grill WM. Modeling the excitability of mammalian nerve fibers: influence of afterpotentials on the recovery cycle. J Neurophysiol. 2002 Feb; 87(2): 995-1006.

TABLE 2a
Electrical parameters of MRG models.

| Parameters | Value* | |
|---|---|---|
| maximum fast Na⁺ conductance g̅_{Naf} | 3.0 | S/cm² |
| maximum slow K⁺ conductance g̅_{Ks} | 0.08 | S/cm² |
| maximum persistent Na⁺ | 0.01 | S/cm² |
| conductance g̅_{Nap} | | |
| nodal leakage conductance g_{L} | 0.007 | S/cm² |
| Na⁺ reversal potential E_{Na} | 50 | mV |
| K⁺ reversal potential E_{K} | −90 | mV |
| leakage reversal potential E_{L} | −90 | mV |
| nodal capacitance Cₙ | 2 | μF/cm² |
| internodal capacitance Ci | 2 | μF/cm² |
| myelin capacitance Cₘ | 0.1 | μF/cm² |
| myelin conductance Gₘ | 0.001 | S/cm² |
| MYSA conductance gₐ | 0.001 | S/cm² |
| FLUT conductance g_{f} | 0.0001 | S/cm² |
| STIN conductance gᵢ | 0.0001 | S/cm² |
| axoplasmic resistivity ρₐ | 70 | Ω · cm |
| periaxonal resistivity ρₚ | 70 | Ω · cm |

*McIntyre CC, Richardson AG, Grill WM. Modeling the excitability of mammalian nerve fibers: influence of afterpotentials on the recovery cycle. J Neurophysiol. 2002 Feb; 87(2): 995-1006.

TABLE 2b
Electrical parameters of MRG models.

| Parameters | Value* | |
|---|---|---|
| maximum fast Na⁺ conductance g̅_{Naf} | 3.0 | S/cm² |
| maximum slow K⁺ conductance g̅_{Ks} | 0.08 | S/cm² |
| maximum persistent Na⁺ | 0.01 | S/cm² |
| conductance g̅_{Nap} | | |
| nodal leakage conductance g̅_{L} | 0.007 | S/cm² |
| Na⁺ reversal potential E_{Na} | 50 | mV |
| K⁺ reversal potential E_{K} | −90 | mV |
| leakage reversal potential E_{L} | −90 | mV |
| nodal capacitance Cₙ | 2 | μF/cm² |
| internodal capacitance Cᵢ | 2 | μF/cm² |
| myelin capacitance Cₘ | 0.1 | μF/cm² |
| myelin conductance Gₘ | 0.001 | S/cm² |
| MYSA conductance gₐ | 0.001 | S/cm² |
| FLUT conductance g_{f} | 0.0001 | S/cm² |
| STIN conductance gᵢ | 0.0001 | S/cm² |
| axoplasmic resistivity ρₐ | 70 | Ω · cm |
| periaxonal resistivity ρₚ | 70 | Ω · cm |

*McIntyre CC, Richardson AG, Grill WM. Modeling the excitability of mammalian nerve fibers: influence of afterpotentials on the recovery cycle. J Neurophysiol. 2002 Feb; 87(2): 995-1006.

TABLE 3
Parameters of the transition rates for Naᵥ 1.1 and Naᵥ 1.6 channels.*

| | | | | | |
|---|---|---|---|---|---|
| B_{hyp} | V_{hyp} | k_{hyp} | B_{dep} | V_{dep} | k_{dep} |

Nav 1.1 channels

| | | | | | | |
|---|---|---|---|---|---|---|
| C1C2 | 0 | 0 | 0 | 18 | −7 | −10 |
| C2C1 | 3 | −37 | 10 | 18 | −7 | −10 |
| C2O1 | 0 | 0 | 0 | 18 | −7 | −10 |
| O1C2 | 3 | −37 | 10 | 18 | −7 | −10 |
| C2O2 | 0 | 0 | 0 | 0.08 | −10 | −15 |
| O2C2 | 2 | −50 | 7 | 0.2 | −20 | −10 |
| O1I1 | 8 | −37 | 13 | 17 | −7 | −15 |
| I1O1 | 0.00001 | −37 | 10 | 0 | 0 | 0 |
| I1C1 | 0.21 | −61 | 7 | 0 | 0 | 0 |
| C1I1 | 0 | 0 | 0 | 0.3 | −61 | −5.5 |
| I1I2 | 0 | 0 | 0 | 0.0015 | −90 | −5 |
| I2I1 | 0.0075 | −90 | 15 | 0 | 0 | 0 |

Nav 1.6 channels

| | | | | | | |
|---|---|---|---|---|---|---|
| C1C2 | 0 | 0 | 0 | 14 | −8 | −10 |
| C2C1 | 2 | −38 | 9 | 14 | −8 | 10 |
| C2O1 | 0 | 0 | 0 | 14 | −18 | −10 |
| O1C2 | 4 | −48 | 9 | 14 | −18 | −10 |
| C2O2 | 0 | 0 | 0 | 0.0001 | −10 | −8 |
| O2C2 | 0.0001 | −55 | 10 | 0.0001 | −20 | −5 |
| O1I1 | 6 | −40 | 13 | 10 | −15 | −18 |
| I1O1 | 0.00001 | −40 | 10 | 0 | 0 | 0 |
| I1C1 | 0.1 | −86 | 9 | 0 | 0 | 0 |
| C1I1 | 0 | 0 | 0 | 0.08 | −55 | −12 |
| I1I2 | 0 | 0 | 0 | 0.00022 | −50 | −5 |
| I2I1 | 0.0018 | −90 | 30 | 0 | 0 | 0 |

*Balbi P, Massobrio P, Hellgren Kotaleski J. A single Markov-type kinetic model accounting for the macroscopic currents of all human voltage-gated sodium channel isoforms. PLoS Comput Biol. 2017 Sep 1; 13(9): e1005737.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A method of designing a waveform shape for blocking neural conduction without inducing an onset response, the method comprising:
identifying an optimized transmembrane voltage trajectory sufficient to induce closed-state inactivation (CSI) in a plurality of voltage-gated sodium channels;
identifying at least one DC blocking waveform from a plurality of candidate waveforms using a global optimization algorithm, wherein the at least one DC blocking waveform drives a voltage profile corresponding to the optimized transmembrane voltage trajectory; and
designing a waveform that blocks neural conduction without inducing an onset response based on the at least one DC blocking waveform.

2. The method of claim 1, wherein the waveform that blocks neural conduction without inducing an onset response is an AC blocking waveform.

3. The method of claim 2, wherein the AC blocking waveform comprises an amplitude envelope defined by the least one DC blocking waveform.

4. The method of claim 1, wherein the optimized transmembrane voltage trajectory is identified using a computation model comprising characteristics of a nerve fiber.

5. The method of claim 4, wherein the characteristics of the nerve fiber are selected from the group consisting of number of nodes of Ranvier, nerve fiber diameter, nerve fiber length, nerve fiber location, types of sodium channels, number of sodium channels, types of potassium channels, number of potassium channels, and degree of myelination.

6. The method of claim 1, wherein the optimized transmembrane voltage trajectory is identified using a global optimization algorithm.

7. The method of claim 6, wherein the global optimization algorithm is selected from the group consisting of a genetic algorithm, a particle swarm algorithm, a simulated annealing algorithm, an ant colony algorithm, an estimation of distribution algorithm, and any combinations and derivations thereof.

8. The method of claim 1, wherein the global optimization algorithm is selected from the group consisting of a genetic algorithm, a particle swarm algorithm, a simulated annealing algorithm, an ant colony algorithm, an estimation of distribution algorithm, and any combinations and derivations thereof.

9. The method of claim 1, wherein the optimized transmembrane voltage trajectory increases nonlinearly from a resting potential to a suprathreshold level.

10. The method of claim 2, wherein the AC blocking waveform is applied at increasing amplitude over time.

11. The method of claim 2, wherein the AC blocking waveform is charge-balanced.

12. The method of claim 2, wherein the AC blocking waveform is biphasic, monophasic, or multiphasic.

13. The method of claim 2, wherein the AC blocking waveform is applied at a frequency of about 5 kHz to about 90 kHz.

14. The method of claim 2, wherein the AC blocking waveform is symmetric and/or rectangular.

15. An AC conduction block waveform that eliminates onset response, wherein the waveform is applied at increasing amplitude over time, and wherein the waveform blocks neural conduction and induces closed-state inactivation (CSI) of voltage-gated sodium channels prior to activation.

16. A system for blocking neural conduction without inducing an onset response, the system comprising:
an electrode sized and configured for implantation in proximity to neural tissue; and
a pulse generator coupled to the electrode, the pulse generator including a power source comprising a battery and a microprocessor coupled to the battery, wherein the pulse generator is capable of applying to the electrode an AC waveform capable of blocking neural conduction and eliminating onset response by inducing closed-state inactivation (CSI) of voltage-gated sodium channels prior to activation.

17. The system according to claim 16, wherein the AC blocking waveform is applied at increasing amplitude over time.

18. The system of claim 16, wherein the AC blocking waveform is charge-balanced.

19. The system of claim 16, wherein the AC blocking waveform is biphasic, monophasic, or multiphasic.

20. The system of claim 16, wherein the AC blocking waveform is applied at a frequency of about 5 kHz to about 90 kHz.

21. The system of claim 16, wherein the AC blocking waveform is symmetric and/or rectangular.

22. A method for blocking neural conduction in a subject using the system of claim 16, the method comprising:
programming the pulse generator to output the blocking waveform at increasing amplitude over time; and
delivering the blocking waveform to the subject without inducing an onset response.
